(19) United States
(12) Patent Application Publication
(10) Pub. No.: US 2026/0354842 A1
(43) Pub. Date: Nov. 12, 2026

(54) BONE RASP BLADE

(71) Applicant: Treace Medical Concepts, Inc., Ponte Vedra, FL (US)

(72) Inventors: Sean F. Scanlan, Jacksonville, FL (US); Jared White, St. Augustine, FL (US); George Sarkees, Ponte Vedra, FL (US)

(21) Appl. No.: 19/578,983

(22) Filed: Mar. 26, 2026

Related U.S. Application Data
(60) Provisional application No. 63/778,184, filed on Mar. 26, 2025.

(51) Int. Cl. A61B 17/16

(52) U.S. Cl. CPC A61B 17/1659; A61B 17/1628

(57) ABSTRACT

An apparatus, system, and method are disclosed for shaping and contouring a bone surface. In some implementations, a rasp blade includes a body extending along a longitudinal axis, with a proximal oscillation mount configured to couple to a powered oscillation handpiece. A foot at the distal end includes a planar inferior side with a plurality of rasp features. The foot connects to the body via an angle feature that positions the foot at an acute inclination. In some embodiments, the foot includes openings for bone debris evacuation, and the inclination angle ranges from about 30 to 60 degrees. A surface contouring blade embodiment features a plurality of teeth configured for controlled abrasion. The apparatus or system may provide a mass moment of inertia tailored for optimal performance. A method of use includes engaging the rasp features against a bone surface to remove a controlled amount of material.

### CROSS REFERENCE TO RELATED APPLICATIONS

This non-provisional patent application claims the benefit of and priority to U.S. Provisional Patent Application No. 63/778,184, filed in the United States Patent and Trademark Office (USPTO) on Mar. 26, 2025. The entire contents of the provisional application are hereby incorporated by reference herein in its entirety.

### TECHNICAL FIELD

The present disclosure relates to surgical devices, systems, instruments, and methods. More specifically, the present disclosure relates to bone rasps for use with powered oscillation handpieces and/or methods of designing and using the same.

### BACKGROUND

Conventional rasps for use with powered oscillation handpieces are often used to prepare bone surfaces for fixation and fusion as a resection heals. These rasps are commonly configured for use between bones at joints or at bone ends and may be used to clear soft tissue from articulating surfaces or fusion sites. Additionally, conventional rasps can create an exposed and bleeding bone surface to promote healing. Many such rasps feature a curved profile to contour exposed bone surfaces at a joint. Consequently, conventional rasps are often designed for specific surgical procedures and anatomical locations, limiting their versatility.

Other rasp tools are configured for use with powered reciprocation handpieces and may be integrated with long extensions to facilitate access to hard-to-reach anatomical areas. However, the distal positioning of these rasps, combined with the length of the extensions, can complicate precise bone removal, particularly between bones at a joint. Limited visibility in such procedures can further hinder surgical accuracy. As a result, existing solutions are often highly specialized, making them inadequate or error-prone for broader orthopedic applications.

Due to the procedure-specific nature of conventional rasps, surgeons may be reluctant to use these tools for general bone contouring and shaping. What is needed is a bone rasp blade that is compatible with a powered oscillation handpiece and configured for effective bone removal from bone surfaces. Further, a need exists for a bone rasp blade designed to couple with the same powered oscillation handpieces used for bone-cutting saw blades, enhancing surgical versatility. Additionally, a bone rasp blade is needed that provides high visibility to enable precise and controlled bone removal for shaping and contouring bones to a desired profile. Finally, there is a need for a bone rasp blade that allows a surgeon to reduce prominences, remove osteophytes, and smooth bone surfaces in preparation for implant placement.

### SUMMARY

The various apparatus, devices, systems, and/or methods of the present disclosure have been developed in response to the present state of the art and, in particular, in response to the problems and needs in the art that have not been met for a rasp or bone contouring tool.

The present disclosure relates to a rasp blade and/or a surface contouring blade designed for shaping and contouring bone surfaces, particularly in surgical applications involving powered oscillation handpieces. These blades incorporate features that facilitate efficient and controlled material removal while maintaining compatibility with conventional oscillating surgical instruments.

In some embodiments, a rasp blade is provided, having a body extending along a longitudinal axis, with a proximal end and a distal end. The proximal end includes an oscillation mount configured to securely couple the rasp blade to a powered oscillation handpiece. The distal end of the rasp blade includes a foot, which includes a superior side and a planar inferior side having a plurality of rasp features designed to engage a bone surface for shaping and contouring. An angle feature connects the foot to the body. The angle feature positions the foot at an acute angle relative to the longitudinal axis to enhance cutting efficiency and control.

In certain embodiments, the superior side of the foot is planar and includes a plurality of openings that extend through the foot to the inferior side. These openings facilitate the evacuation of bone debris during use, improving visibility and reducing clogging of the rasp features. Some variations include openings positioned between at least two rasp features to optimize debris removal.

The angle feature of the rasp blade may include an inclination curve that provides a smooth transition between the body and the foot. The inclination angle of the foot relative to the longitudinal axis may range from about 30 degrees to about 60 degrees, with some implementations specifically having an inclination angle of 45 degrees to optimize performance.

The rasp blade may be engineered to provide a specific mass moment of inertia at the rasp features, typically between about 725 gmm² to 750 gmm², ensuring stability and efficient energy transfer during oscillation. The body may also include a shaped opening positioned between the foot and the oscillation mount to influence weight distribution and mechanical properties. In some embodiments, the shaped opening has a distal end with a larger diameter than the proximal end, further refining the blade's dynamic performance.

A surface contouring blade is also disclosed, having a body extending along a longitudinal axis with a proximal end that includes an oscillation mount for coupling to a powered oscillation handpiece. The distal end of the body features a superior side and a planar inferior side having a plurality of teeth arranged for contouring a bone surface. An inclination feature tilts the distal end at an acute angle relative to the longitudinal axis, enabling improved control during contouring procedures.

In certain embodiments, the inclination feature includes an inclination curve that connects the distal end of the body to the body itself. This curve is configured to bias the distal end to return to its acute angle when the teeth are pressed against a bone surface, allowing for consistent engagement and predictable performance. The plurality of teeth may be designed to include coarse rasp features and fine rasp features, with the coarse rasp features having a greater tooth height or spacing than the fine rasp features.

At least one tooth of the surface contouring blade may have a base connected to a tip by four sides, where the four sides converge at the tip to form an abrading feature. The geometric arrangement of the sides may include obtuse and acute angles to enhance cutting efficiency while minimizing resistance.

The present disclosure may include a method for remediating a bone condition in a patient. The method includes determining the need for bone surface contouring in a surgical procedure and coupling a powered oscillation handpiece to a rasp blade. The rasp blade is positioned such that its rasp features engage a bone surface, and upon activation of the handpiece, a controlled amount of bone is removed. In some implementations, a first rasp blade with coarse rasp features can be used initially, followed by a second rasp blade having fine rasp features to refine the surface.

These embodiments provide an efficient and controlled approach to shaping and contouring bone surfaces, ensuring compatibility with existing surgical instruments while enhancing precision and usability. Various modifications and alternative designs are contemplated, allowing for adaptations based on surgical requirements and patient-specific conditions.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1 is a perspective view of a rasp blade according to one embodiment.

FIGS. 2A-2G illustrate views of the rasp blade of FIG. 1, according to one embodiment.

FIG. 3A is a side view of a rasp blade according to one embodiment.

FIG. 3B is a superior side view of a rasp blade according to one embodiment.

FIG. 4 is a perspective view of a rasp blade according to one embodiment.

FIG. 5A is a cross-section view of the surface contouring blade of FIG. 4 taken along lines 5-5 according to one embodiment.

FIG. 5B is close up view of teeth of a surface contouring blade according to one embodiment.

FIG. 6 is an inferior side view of a rasp blade according to one embodiment.

FIG. 7A is a perspective view of a rasp blade according to one embodiment.

FIG. 7B is a distal end view of a rasp blade according to one embodiment.

FIG. 8 is a flowchart diagram depicting a method for remediating a condition, according to one embodiment.

FIG. 9 illustrates an exemplary system configured to provide fixation to two bones using a rasp blade according to one embodiment.

FIGS. 10A & 10B illustrate views of a rasp blade according to an embodiment.

FIG. 11A illustrates a side view of a rasp blade shown in FIGS. 10A & 10B.

FIG. 11B illustrates an inferior side view of a rasp blade shown in FIGS. 10A & 10B.

FIG. 12A-12C illustrate views of a rasp blade according to an embodiment.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region.

“Bone surface” refers to a surface of a bone.

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g., PLLA) and/or others. In one embodiment, a body may include a housing or frame, or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “end” refers to a part or structure of an area or span that lies at the boundary or edge. An end can also refer to a point that marks the extent of something and/or a point where something ceases to exist. An end can also refer to an extreme or last part lengthwise of a structure or surface.

“Longitudinal axis” or “Long axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

“Oscillation mount” refers to a mount device, apparatus, component, module, part, piece, mounting structure, or the like configured, designed, and/or engineered to facilitate the attachment or coupling of a tool, blade, apparatus, device, or component to an oscillation handpiece (powered or unpowered). The oscillation mount may include features for secure coupling, stability, and compatibility with various tool configurations.

“Rasp blade” refers to a tool, apparatus, or device configured to engage with an oscillation handpiece for modifying or removing material from an object. The object may include biological tissue, synthetic materials, or other surfaces requiring reshaping, contouring, or material removal.

A rasp blade operates by oscillating through a defined arc, interacting with the target object to remove, shape, or refine its surface. It may include one or more rasping features that engage with the object through cutting, abrading, tearing, or scraping actions.

A rasp blade may be adaptable to various oscillation handpieces, including those designed for oscillating saw blades or other tool attachments. It may be configured to remove material at different rates, from aggressive material reduction to fine precision adjustments, allowing the user to control the degree of modification.

“Powered oscillation handpiece” refers to a motor-driven surgical instrument designed to generate repeated angular displacement about a pivot axis or axis of rotation for use in osteotomies and other precision cutting, shaping, or material removal procedures. The handpiece transmits oscillatory motion to an attached tool—such as a rasp blade, saw blade, burr, or other cutting instrument—allowing for controlled engagement with bone or other surgical targets.

A powered oscillation handpiece may incorporate electromechanical, pneumatic, or other drive mechanisms to provide variable-speed oscillation, enabling precise control over depth, speed, and force of material removal. The device is designed for compatibility with various surgical tool attachments, including those used for bone shaping, contouring, resection, or controlled excision.

In certain embodiments, the powered oscillation handpiece may include ergonomic handling features, vibration-dampening structures, or integrated cooling systems to enhance surgical precision and reduce procedural fatigue.

As used herein, “side” refers to a structure or part of a structure including but not limited to one of a longer bounding surfaces or lines of an object especially contrasted with the ends, a line or surface forming a border or face of an object, either surface of a thin object, a bounding line or structure of a geometric figure or shape, and the like.

A side can also refer to a geometric edge of a polygon (two-dimensional shape) and/or a face or surface of a polyhedron (three-dimensional shape).

Side can also refer to a location on a structure. For example, a side can be a location on a structure at, or near, a furthest position away from a central axis of the structure. As used herein, the term “side” can include one or more modifiers that define and/or orient and/or distinguish the side of an object from others based on based on where and/or how the object is deployed within or in relation to a second object.

For example, in the context of an implant or tool for use with a patient, sides of the implant may be labeled based on where the sides are relative to the patient when the implant is deployed. As one example, an “anterior side” of an implant, instrument, anatomical structure, or other structure refers to a side that is anterior to other sides of the structure in relation to a patient when the structure is deployed in the patient. As another example, in the context of an instrument used with a patient, sides of the instrument may be labeled based on where the sides are when the instrument is being used for its purpose. As one example, a “front side” of an instrument refers to a side that is facing a user of the instrument when the instrument is in use.

As used herein, “feature” refers to a distinctive attribute or aspect of something. A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “rasp feature,” “attachment feature,” “alignment feature,” “securing feature,” “placement feature,” “protruding feature,” “engagement feature,” “disengagement feature,” “resection feature”, “guide feature”, “alignment feature,” and the like.

“Rasp feature,” as used herein, refers to a feature and/or a structural component of a cutting instrument, such as a rasp and/or rasp blade, or the like configured to abrade, grind, shape, contour, or refine surfaces during a procedure. The rasp feature enhances the cutting instrument's versatility by enabling both precise cutting and controlled surface sculpting.

In some embodiments, the rasp feature comprises a plurality of raised protrusions, serrations, ridges, or teeth disposed along at least a portion of an instrument's working surface, such as a planar surface, an edge, a distal end, a distal surface, and/or a lateral face thereof. These protrusions may be arranged in a pattern, such as linear rows, arcuate rows, a grid, or an irregular array, and are sized and shaped to remove material incrementally through frictional engagement, thereby smoothing and/or contouring the surface rather than solely incising the surface. For example, the rasp feature may include teeth or protrusions with a height ranging from approximately about 0.1 mm to about 2 mm and a spacing of approximately about 0.5 mm to about 5 mm, though other dimensions are contemplated depending on the intended application.

Alternatively, the rasp feature may be embodied as a textured surface, such as a roughened, etched, or grit-coated area, applied to the instrument's working surface to facilitate abrasion through microscopic or macroscopic surface irregularities. Such a textured surface may be achieved, for instance, through chemical etching, laser texturing, or the application of an abrasive coating, providing a uniform or patterned roughness with a surface roughness average (Ra) ranging from approximately 0.5 μm to 50 μm, depending on the desired abrasiveness.

The rasp feature, whether formed as protrusions, serrations, teeth, or a textured surface, may be integrally formed with the instrument body, such as through machining, molding, or surface treatment, or may be a distinct element affixed thereto, such as a coated abrasive layer. This flexibility in construction allows the rasp feature to be adapted to various manufacturing processes and instrument designs, supporting a range of applications where both cutting and/or surface refinement are desired.

“Abrading feature,” as used herein, refers to a feature and/or a structural component of an instrument, such as an abrader, abrading blade, burr, file, rasp, rasp blade, or similar tool, configured to remove, smooth, shape, contour, or refine surfaces through abrasion during a procedure. The abrading feature enhances the instrument's functionality by enabling controlled material removal and surface modification, which may be used to complement cutting and shaping operations.

In some embodiments, the abrading feature comprises a plurality of raised elements, abrasive edges, edges, teeth, ridges, and/or particulate-coated regions disposed along at least a portion of the instrument's working surface, such as a planar surface, an edge, a distal end, a distal surface, and/or a lateral face thereof. These elements may be arranged in a linear, arcuate, radial, grid-like, or irregular pattern and are dimensioned to incrementally remove material through frictional engagement. The abrading feature may smooth or contour a surface rather than incising it, enabling controlled reshaping without excessive material loss.

For example, the abrading feature may include raised abrasive elements with a height ranging from approximately 0.1 mm to 2 mm and a spacing of approximately 0.5 mm to 5 mm, though other dimensions are contemplated based on the intended application. Alternatively, the abrading feature may be embodied as a textured or grit-coated surface, including a roughened, etched, micro-patterned, or abrasive-coated area applied to the instrument's working surface to enhance material removal efficiency. Such a textured surface may be achieved through chemical etching, laser structuring, abrasive blasting, or the application of bonded abrasive materials, yielding a uniform or patterned roughness with a surface roughness average (Ra) ranging from approximately 0.5 μm to 50 μm, depending on the desired level of abrasiveness.

The abrading feature, whether formed as raised elements, ridges, abrasive teeth, particulate coatings, or a textured surface, may be integrally formed with the instrument body through machining, molding, laser structuring, or deposition processes or may be a separately applied or affixed element, such as an abrasive-coated layer, bonded grit structure, or modular insert. This flexibility in construction allows the abrading feature to be adapted to various manufacturing processes, material compositions, and instrument designs, supporting a range of applications where controlled abrasion, contouring, and surface refinement are required.

“Angle feature” refers broadly to a structural component of a cutting instrument that connects and/or positions a cutting face, cutting structure, abrading feature, contouring feature(s), rasp feature(s), or the like relative to the cutting instrument's main body and/or operational axis. In one embodiment, an angle feature can be configured to orient such features at an acute angle relative to a longitudinal axis of a cutting instrument's main body to facilitate effective engagement with a target surface during a procedure (e.g., a surgical procedure), enhancing the cutting instrument's versatility across a range of applications, including but not limited to surgical, industrial, or material-shaping tasks. This angle feature may take various forms to achieve the desired angular orientation, accommodating diverse designs and implementations while ensuring functional alignment with the instrument's intended purpose.

In certain embodiments, such as with a rasp blade configured for shaping a bone surface, the angle feature can refer to a structural component that connects a body to a foot and can establish an acute angle between a planar inferior side of the foot and a longitudinal axis of the body. The angle feature can be configured to position the foot relative to the body such that the rasp features on the planar inferior side can effectively engage and shape a bone surface during operation, such as when coupled to a powered oscillation handpiece via an oscillation mount. This angle feature may be implemented in various forms to achieve the desired angular orientation, including, but not limited to, a curved transition, a linear segment, a stepped configuration, a flexible linkage, and/or a pivotable junction, each adapted to transmit oscillatory motion from the body to the foot while maintaining structural integrity.

In some embodiments, the angle feature may comprise a curved transition, such as an arcuate bend or fillet, integrally formed between the body and the foot, providing a smooth shift in orientation with a radius of curvature that may range, for example, from approximately 2 mm to 10 mm, depending on the intended application. Alternatively, or in addition, the angle feature may be embodied as a linear segment, such as an angled arm or extension, connecting the body to the foot at a fixed acute angle. The acute angle provided by the angle feature may vary, for instance, from about 30 degrees to about 60 degrees relative to the longitudinal axis, to optimize access to target surfaces.

In other embodiments, the angle feature may include a stepped configuration, characterized by one or more discrete angular shifts or terraces between the body and the foot, such a configuration may enhance stability and/or provide multiple working angles within a single structure. Additionally, the angle feature may be implemented as a flexible linkage, such as a resilient material or segmented joint, allowing dynamic flexure during use, or as a pivotable junction, such as a hinge or ball-and-socket mechanism with a pivot lock mechanism, permitting adjustable angular positioning of the foot relative to the body, with an angular range that may span, for example, from 0 degrees (aligned with the longitudinal axis) to 89 degrees.

The angle feature may be integrally formed with the body and/or foot, such as through machining, molding, or forging of a unitary material (e.g., stainless steel, titanium), or it may comprise a separate component affixed thereto, such as by welding, adhesive bonding, and/or mechanical fastening. The specific design of the angle feature, including its shape, size, and angle, may be selected based on factors such as the type of surface to be engaged, the motion characteristics of the cutting instrument, and/or ergonomic considerations for the user. Regardless of its form, the angle feature ensures that the cutting face, abrading feature, rasp feature(s), or similar elements are oriented to cut, abrade, grind, shape, or refine surfaces effectively, enhancing the instrument's utility across diverse procedures.

“Foot” refers to a structural feature of a device, component, apparatus, or system that extends from and/or is connected to a larger device, component, body, apparatus, system, a leg structure, or the like and is configured to engage, support, contact, modify, or apply force to a surface, object, or medium. A foot can facilitate stability, positioning, material interaction, force transmission, or other functional engagements between the device and an external structure. The term “foot” can encompass any structure performing analogous functions to a biological foot, including engagement, stabilization, movement control, and material modification.

In various embodiments, a foot may comprise one or more surfaces, edges, or contact points that interact with a target object or surface. The foot and/or its surfaces, edges, or contact points may be planar, contoured, curved, segmented, flexible, or rigid, and may be positioned at various angles relative to a leg and/or body of a device, component, apparatus, or system. In certain configurations, the foot may be integrally formed with, removably attached to, or movably connected to a leg and/or device to allow for articulation, pivoting, or controlled motion.

A foot may perform one or more functions, including but not limited to: Load distribution and surface engagement, such as stabilizing a cutting or abrading tool against a material surface; Material modification, such as incorporating rasping, cutting, or abrasive features to remove or reshape material; Force application or transmission, such as controlling contact pressure during oscillation or movement; Pivoting or articulated movement, allowing the foot to adjust dynamically during operation.

Examples of a foot include, but are not limited to, a rasp blade foot configured to oscillate against a bone surface, where a planar inferior side includes rasping features for material removal, a surgical saw foot providing a stable contact surface to guide an oscillating or reciprocating blade, a robotic gripper foot designed to stabilize or engage a surface through pressure or frictional contact, a prosthetic foot that mimics the functionality of a biological foot for weight distribution and movement, or the like.

“Planar” refers to a surface, structure, or feature that extends predominantly along a flat geometric plane. A planar surface may be entirely flat or may include minor variations, curvatures, or surface features, provided that it retains an overall substantially flat configuration relative to an adjacent structure, reference axis, or functional requirement.

In various embodiments, a planar surface may be oriented horizontally, vertically, or at an angle relative to another component. A planar surface may include textured, perforated, or contoured features, such as ridges, grooves, rasp features, or recesses, while maintaining an overall flat form. A planar surface may function as a supporting, contacting, guiding, or engaging surface, depending on the application.

Examples of planar features include a planar inferior surface of a rasp blade foot defining a substantially flat engagement surface with rasps features, a planar guide surface of a surgical cutting tool ensuring controlled movement along a fixed plane, a planar electrode contact area in an electronic device facilitating uniform electrical interaction, and a planar bearing surface in a mechanical assembly allowing for stable load distribution.

As used herein, the term “planar” encompasses surfaces that are predominantly flat in structure while permitting minor deviations, texturing, or functional modifications consistent with their intended use.

“Shaped opening” refers to an opening that has a certain shape. A shaped opening can be two-dimensional or three-dimensional and can have a variety of regular or irregular geometric shapes and/or cross-sectional shapes, including, but not limited to a rectangle, a square, triangle, or other polygon, as well as a circle, an ellipse, an ovoid, or other circular or semi-circular shape. In one embodiment, a shaped opening may have one shape when a structure that includes the shaped opening is in a first configuration and a second shape, different from the one shape, when the shaped opening is in a second configuration.

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.” “Evacuation” refers to the process of removing bone debris, bone chips, or other particulate material from the cutting face or set of rasp features of a surgical tool during operation. Evacuation may occur through passive or active mechanisms to enhance cutting efficiency, maintain clear visibility, and reduce heat buildup or clogging. In certain embodiments, evacuation may be facilitated by tooth or rasp feature geometry, including gullet spacing, serrations, or fluting, which directs bone debris away from the cutting face; oscillatory or reciprocating motion, which aids in self-clearing of bone material from engagement surfaces; integrated fluid irrigation systems, such as saline or cooling agents, which assist in flushing debris from the cutting interface; vacuum-assisted suction mechanisms, which actively remove bone particles from the cutting region; openings, ports, or channels specifically designed and/or configured to facilitate evacuation, which may include apertures within the blade, fenestrations in the rasp surface, or grooves along the cutting edge to promote continuous removal of debris. As used herein, “evacuation” encompasses any mechanism or structural design feature that promotes the removal, displacement, or clearing of bone debris from the cutting face or rasp features, whether by mechanical action, fluid assistance, suction, or integrated openings configured for debris management.

“Bone debris” refers to particulate material generated during the cutting, shaping, or modification of bone using surgical instruments such as osteotomy blades, rasp blade, contouring blades, osteotomy rasps, saws, drills, or the like. Bone debris may include bone chips, bone dust, fragments, or other dislodged material resulting from mechanical interaction between a cutting tool or rasping tool or contouring tool and bone tissue. In certain embodiments, bone debris may vary in size, composition, and morphology, depending on factors such as the cutting speed, tool geometry, bone density, and applied force. Bone debris may consist of cortical bone particles, cancellous bone fragments, or a combination thereof, and may accumulate at or near the cutting interface/face, and may affect cutting efficiency, visibility, and/or heat dissipation. As used herein, “bone debris” encompasses any solid byproduct of bone modification that may require evacuation, removal, or management to maintain optimal surgical performance, ensure procedural accuracy, or facilitate bone healing.

“Inclination angle” refers to a type of angle that directs a structural component, such as a foot, cutting face, cutting teeth, one or more rasp features or the like, upward relative to the longitudinal axis of a cutting or rasping instrument's main body. The inclination angle may be a subset of an acute angle established by an angle feature, and the inclination angle may govern the elevated positioning of the structural component relative to a cutting instrument's main body to optimize engagement with a target surface. In certain embodiments, the inclination angle is selected to enhance procedural access, improve cutting efficiency, improve visibility, and/or control material removal rates. For example, in a rasp blade configured for shaping a bone surface, the inclination angle can ensure that rasp features (such as on a foot) are angled upward relative to the longitudinal axis, positioning the rasp features to engage a bone surface at an orientation that facilitates controlled shaping or contouring and visibility. The inclination angle may vary depending on the instrument's intended use, with typical inclination angles ranging from about 20 degrees to about 60 degrees relative to the longitudinal axis. The inclination angle can be structurally defined within an angle feature and can serve to maintain an optimal working orientation during operation, such as when the instrument is coupled to a powered oscillation handpiece. As used herein, “inclination angle” refers to an angle that directs a foot, cutting face, cutting teeth, rasp feature, cutting surface, working feature or the like upward relative to the longitudinal axis, ensuring proper functional alignment and engagement with a target surface.

“Inclination curve” refers to a structural transition that defines a smooth, continuous, or graded curvature between two connected components of a device, mechanism, or instrument. The inclination curve establishes an angular relationship between the connected components and may function to position, support, or align one component relative to another while maintaining structural integrity and facilitating engagement with a target surface. The inclination curve may serve to transition between different planes, surfaces, or orientations, providing a gradual or progressive shift that may enhance mechanical performance, stability, or force distribution.

The radius of curvature of the inclination curve may vary based on mechanical and procedural considerations, with typical values ranging from 2 mm to 15 mm, depending on the degree of angular shift required for the rasp blade's engagement with a target surface. In some embodiments, the inclination curve may provide a gradual transition between the body and the foot that may reduce mechanical stress concentrations, enhance oscillatory motion transmission, and improve cutting or abrading stability when the rasp blade is coupled to a powered oscillation handpiece.

As used herein, “inclination curve” may refer to a curved or non-linear structural transition within or as part of an angle feature, wherein the inclination curve establishes an angular relationship between a body and a foot to facilitate positioning, stability, and operational efficiency of a cutting instrument. In the context of an angle feature, an inclination curve refers to a structural transition that can connect a body to a foot of a cutting or rasping instrument, wherein the inclination curve defines a smooth, continuous, or graded curvature that may establish an angular orientation between the body and the foot.

The inclination curve may function to position, support, and align the foot relative to the body while maintaining structural integrity and facilitating engagement with a target surface. In certain embodiments, the inclination curve can define a non-linear transition that gradually or progressively shifts the orientation of the foot relative to a longitudinal axis of the body. The curvature of the inclination curve may be arcuate, parabolic, filleted, or otherwise contoured, depending on the desired angular transition and operational requirements.

The inclination curve may be integrally formed as part of an angle feature or in place of an angle feature through machining, molding, forging, or additive manufacturing, or may be defined by a separately affixed structural component.

“Mass moment of inertia”, “Moment”, or “Moment of inertia” refers to a measure of an object's resistance to angular acceleration about a specific axis of rotation. The mass moment of inertia is determined by the distribution of an object's mass relative to the axis of rotation, where an increase in mass at a greater distance from the axis results in a higher moment of inertia. The mass moment of inertia may be used to quantify how mass is spatially arranged and how that arrangement affects the rotational dynamics of an object. The mass moment of inertia is typically expressed in units of kilogram meter squared.

As used herein, a “pivot axis” refers to an axis about which a structure pivots or rotates.

“Moment arm” refers to the perpendicular distance between a specified axis of rotation and the line of action of a force applied to a body. The moment arm determines the effectiveness of the applied force in generating torque about the axis of rotation, where a greater moment arm results in a proportionally larger torque for the same applied force. The length of the moment arm may depend on the spatial relationship between the force application point and the rotational axis, and may be influenced by factors such as mechanical linkages, joint configurations, or force orientation. The moment arm is commonly used in mechanical and biomechanical analysis to assess torque generation and load distribution in various systems, including structural mechanics, robotics, and human movement. The moment arm is typically expressed in units of length, such as meters in the International System of Units or feet in the Imperial system.

In the context of an oscillation mount that connects a rasp blade to an oscillation handpiece, the moment arm refers to the perpendicular distance between the axis of oscillation (pivot axis) and a point or points where forces are transferred to the rasp blade at the contact points or interfaces of the oscillation mount.

The moment arm may influence how torque generated by the oscillation handpiece is transmitted to the rasp blade, affecting bone removal efficiency, rasp blade stability during operation, and the structural integrity of the oscillation mount. A longer moment arm may result in greater torque being applied to the rasp blade, which may enhance material removal efficiency but also increase mechanical loading at the oscillation mount, potentially leading to structural failure. A shorter moment arm may reduce torque output while improving stability and structural reliability of the oscillation mount but may require increased oscillation amplitude or force application to achieve the desired bone resection or shaping.

The mass of the rasp blade, combined with a larger moment arm, may introduce greater inertial effects, influencing oscillatory motion and load distribution at the oscillation mount. The moment arm may be affected by factors such as the mounting configuration of the rasp blade, the positioning of the cutting or abrading surfaces relative to the oscillation axis, and the dynamic forces generated during operation.

“Surface contouring blade” refers to a cutting, rasping, or abrading instrument configured to modify, shape, or refine a target surface through controlled and/or precise material removal. A surface contouring blade may be designed to engage a variety of materials, including biological tissues such as bone, cartilage, soft tissue, or the like as well as non-biological materials in industrial or manufacturing applications. A surface contouring blade may include one or more cutting edges, a cutting face, set of cutting or abrading teeth, abrading surfaces, rasp features, or contouring structures arranged to facilitate precise and controlled surface modification.

In certain embodiments, a surface contouring blade may be configured for use with a powered oscillation handpiece, rotary tool, or manual instrument, wherein the motion of the surface contouring blade influences the manner in which material is removed. The shape, size, and orientation of the surface contouring blade and its cutting or abrading features may be selected to optimize engagement with a target surface, ensuring controlled depth of cut, uniformity, and procedural accuracy.

A surface contouring blade may include specialized geometries, such as curved, angled, or stepped configurations, to accommodate specific contouring requirements. A surface contouring blade may be manufactured from various materials, including but not limited to stainless steel, titanium, carbide, or composite materials, selected based on factors such as durability, cutting efficiency, and resistance to wear. The design of a surface contouring blade may be tailored for specific applications, such as orthopedic surgery, dental procedures, woodworking, or precision machining, where controlled surface modification is required.

“Tooth” or “teeth” refers to a structural feature of a cutting, rasping, or abrading tool that is configured to engage, cut, tear, abrade, or otherwise modify a target material through mechanical interaction. A tooth may be arranged singularly or in a plurality along a cutting edge, along a cutting face, on an abrading surface, and/or within a rasp feature set. A tooth and may be structured, sized, or configured, to optimize material removal efficiency, cutting precision, abrading precision, and/or surface contouring. A tooth may be shaped, arranged, or functionally similar to biological teeth found in animals or humans, but may also encompass artificial or engineered structures specifically designed for cutting, abrading, or shaping applications.

A tooth may include a tooth base, a point, a face, at least one side, and/or a thickness measured from one side to an opposite side of the tooth. The tooth base refers to the portion of the tooth that connects to, or is supported by, a blade, a body, a surface, or a substrate. The point refers to the terminal end of the tooth, which may come to a sharp tip, may resemble a chisel, or may taper into an edge to facilitate material engagement. The face of the tooth includes a surface that contacts the target material. A tooth face may have a rake angle, which can influence the cutting, shearing, or abrading interaction of the tooth with the target material. The at least one side or plurality of sides of the tooth define its lateral boundaries and may contribute to the overall cutting or abrading geometry. The thickness of the tooth is measured between opposing sides and may determine the durability, rigidity, and cutting efficiency of the tooth. A tooth height is measured from the point to the tooth base.

A tooth may be made from a variety of materials, including but not limited to stainless steel, titanium, carbide, ceramic, composite materials, polymers, biocompatible materials, or other engineered substances selected for durability, cutting performance, and wear resistance. A tooth may vary in design based on application-specific requirements and may include serrated, chisel-shaped, hooked, beveled, offset, or other specialized geometries to optimize cutting, abrading, or contouring operations.

As used herein, a “deploy” or “deployment” refers to an act, action, process, system, method, means, or apparatus for inserting an implant or prosthesis into or onto a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases, implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like.

“Fixation,” “fixation device,” “fastener,” or “fastener system” refers to an apparatus, instrument, structure, device, component, member, system, assembly, step, process, or module that is structured, configured, designed, arranged, or engineered to connect, join, engage, or couple two or more structures, either permanently or temporarily. The connected structures may be manmade and/or biological and may include hard tissues such as bone, teeth, or similar materials, as well as soft tissues such as ligaments, cartilage, tendons, or similar biological structures. In certain embodiments, fixation or fastening serves to secure two structures in a desired position and/or orientation, redistribute load or stress, maintain a desired level of tension or compression, and/or reduce relative motion between connected components.

A fixation device or fastener may be made of metal, plastic, composite materials, metal alloys, plastic composites, biocompatible materials, biodegradable materials, or other suitable materials. In some embodiments, a fixation device or fastener may be part of a fastener system that includes two or more structures that work together to function as a fastening mechanism. For example, a fastener system may include a rod or shaft having external threads and an opening or bore within another structure having corresponding internal threads configured to engage the external threads of the rod or shaft.

Fixation devices and fasteners may be used in internal or external fixation applications and may include, but are not limited to: screws, bone screws, set screws, rivets, bolts, nails, pins, Kirschner wires (K-wires), anchors, bone anchors, plates, bone plates, posts, thumb screws, nuts, intramedullary nails, rods, implants, sutures, soft sutures, soft anchors, tethers, interbody cages, fusion cages, staples, bone staples, hook-and-loop mechanisms, snaps, and similar structures. In certain embodiments, a fastener may include an adjective identifying an object or structure that the fastener is particularly configured, designed, or engineered to engage, connect, or couple with. For example, a “bone fastener” may refer to an apparatus for joining or connecting one or more bones, one or more bone portions, soft tissue and a bone or bone portion, hard tissue and a bone or bone portion, or an apparatus and a bone or portion of bone.

In certain embodiments, a fastener may be a temporary fastener, meaning that the fastener is configured to serve a fastening function for a relatively short period of time. A temporary fastener may be used until another procedure or operation is completed and/or until a particular event occurs. A temporary fastener may be designed for removal by a user or may be configured to disengage due to an external event, structural change, or mechanical interaction.

“Tissue” refers to a structure that makes up a one or more anatomical structures of a patient. Tissue can be soft tissue or hard tissue. “Soft tissue” refers to tissue of a patient. Examples of soft tissue include but are not limited to skin, ligament, tendon, fascia, fat muscle, fibrous tissue, blood vessels, lymph vessels, brain tissue, and/or nerves. “Hard tissue” refers to any human tissue that is not soft tissue. Examples of hard tissue include bone, teeth, tooth enamel, dentin, cementum, cartilage, or the like.

As used herein, “osteotomy procedure” or “surgical osteotomy” or “osteotomy” refers to a surgical operation in which one or more bones are cut to shorten or lengthen them or to change their alignment. The procedure can include removing one or more portions of bone and/or adding one or more portions of bone or bone substitutes. As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

As used herein, a “base” refers to a main or central structure, component, or part of a structure. A base is often a structure, component, or part upon which, or from which other structures extend into, out of, away from, are coupled to, or connect to. A base may have a variety of geometric shapes and configurations. A base may be rigid or pliable. A base may be solid or hollow. A base can have any number of sides. In one embodiment, a base may include a housing, frame, or framework for a larger system, component, structure, or device. In certain embodiments, a base can be a part at the bottom or underneath a structure designed to extend vertically when the structure is in a desired configuration or position. Certain bones such as a metatarsal bone can include a base as one structural component of the bone.

“Visibility” refers to a state of feature, aspect, structure, or thing being capable of being observed, seen, or perceived.

As used herein, a “protrusion” refers to a structure or portion of a structure that protrudes or extends from at least one other structure such as a surface of the at least one other structure. Generally, the other structure is connected to, or in contact with, the protrusion.

“Set” refers to a collection of objects. A set can have zero or more objects in the collection. Generally, a set includes one or more objects in the collection.

“Fusion” refers to a natural process of bone growth and generation in which two separate bones and/or bone fragments grow together as new bone grows when the two separate bones and/or bone fragments contact each other. Often, fusion is facilitated by compression of the two separate bones and/or bone fragments towards each other.

As used herein, a “resection” refers to a method, procedure, or step that removes tissue from another anatomical structure or body. A resection can include an osteotomy that cuts through a bone or other tissue because the osteotomy still removes at least a minimal amount of tissue. A resection is typically performed by a surgeon on a part of a body of a patient. A resection is one type of osteotomy. Resection may be used as a noun or a verb. In the verb form, the term is “resect” and refers to an act of performing, or doing, a resection. Past tense of the verb resect is “resected”.

As used herein, a “condition” refers to a state of something with regard to its appearance, quality, or working order. In certain aspects, a condition may refer to a patient's state of health or physical fitness or the state of health or physical fitness of an organ or anatomical part of a patient. In certain embodiments, a condition may refer to an illness, pain, discomfort, defect, disease, or deformity of a patient or of an organ or anatomical part of a patient.

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region. “Malrotation” refers to a condition in which a part, typically a part of a patient's body has rotated from a normal position to an unnormal or uncommon position.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Cortex” refers to an area of bone that extends from an external surface of the bone towards a center part of the bone. The cortex is typically comprised of cortical bone.

“Cut surface” refers to a surface of an object that is created or formed by the removal of one or more parts of the object that includes the original surface. Cut surfaces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface can include manual, mechanical, motorized, hydraulic, automated, robotic, and the like. In certain embodiments, the cut surface(s) are planar.

“Cutting tool” refers to any tool that can be used to cut or resect another object. In particular, a cutting tool can refer to a manual or power tool for cutting or resecting tissue of a patient. Examples of cutting tools include, but are not limited to, a burr, an oscillating saw, a reciprocating saw, a grater saw, a drill, a mill, a side-cutting burr, or the like.

“Cut face” refers to a surface, region, or portion of a cutting tool that is defined by and/or results from the arrangement, orientation, and interaction of one or more cutting teeth, cutting edges, and/or cutting features. The cut face may be formed by a collective engagement of cutting elements (or subset of cutting elements) with a target material, influencing the characteristics of material removal, surface finish, and cutting performance. The geometry, shape, and configuration of the cut face may vary based on factors such as the positioning and spacing of cutting teeth, the rake and engagement angles of the cutting features, how a cutting tool is operated and/or the intended cutting application. Tools or instruments incorporating a cut face include, without limitation, wedge osteotomy blades, rasp blades, saw blades, milling cutters, surgical cutting tools, burrs, and other cutting or abrading instruments configured for controlled material removal.

“Face” refers to a surface, portion, or region of a cutting tooth or similar cutting structure designed or configured to interact with, engage, cut, abrade, or remove material from a target object. The face typically extends from a cutting edge or tip of the tooth toward a base or body of the tooth, and may be angled, contoured, planar, or otherwise shaped to achieve specific cutting or abrading characteristics. The geometry and characteristics of a face may vary based upon factors such as desired cutting efficiency, material to be cut, and overall tooth performance. A face may be defined, characterized, or measured relative to one or more reference dimensions, axes, surfaces, or geometric or functional features of the tooth or structure on which it resides. Components incorporating teeth having faces include, without limitation, wedge osteotomy blades, rasp blades, saw blades, milling cutters, surgical cutting tools, machining tools, and other cutting or abrading instruments configured for material shaping, modification, or removal.

“Coarse rasp feature” refers to a rasp feature or a set of rasp features that are configured to remove a target material at a faster rate than a non-coarse rasp feature. A coarse rasp feature may remove larger portions of the target material due to increased spacing, depth, or aggressiveness of the rasp feature. The material removal may occur in larger fragments, chips, or sections, depending on the geometry of the rasp feature and the properties of the target material. A coarse rasp feature may be used for applications requiring rapid material removal and may be selected based on the desired balance between speed and surface finish quality.

“Fine rasp feature” refers to a rasp feature or a set of rasp features that are configured to remove a target material at a slower rate than a coarse rasp feature or non coarse rasp feature while producing a smoother surface finish. A fine rasp feature may remove smaller portions of the target material due to decreased spacing, shallower depth, or less aggressive geometry of the rasp structure. The material removal may occur in finer particles, shavings, or dust, depending on the geometry of the rasp feature and the properties of the target material. A fine rasp feature may be used for applications requiring precise material removal and improved surface refinement and may be selected based on the desired balance between cutting efficiency and surface finish quality.

“Contour” refers to an outline representing or bounding a shape or form of an object. Contour can also refer to an outside limit of an object, area, or surface of the object.

“Surface contouring” refers to a process, technique, or action that modifies, shapes, or refines a target material by removing, displacing, compressing, or otherwise altering portions of the material to achieve a desired surface profile and/or contour. Surface contouring may involve controlled removal or deformation of material to form specific geometric features, textures, or patterns. The modifications may include forming planar surfaces, curved surfaces, angled surfaces, stepped surfaces, recessed features, raised features, or any other structural variation in the target material.

Surface contouring may be performed using cutting, abrading, rasping, grinding, carving, shaving, pressing, or similar mechanical, chemical, thermal, or other material-altering techniques. The process may be applied to a wide range of materials, including biological tissues such as bone, cartilage, or soft tissue, as well as non-biological materials such as metals, polymers, ceramics, composites, or natural materials. Surface contouring may be executed manually, semi-automatically, or automatically using tools, instruments, machines, or robotic systems specifically configured to modify the surface of the target material in a controlled manner.

“Surgical procedure” refers to a process, method, or series of steps performed using medical instruments, devices, or techniques to diagnose, treat, address, modify, repair, or remove biological tissues in a controlled manner. A surgical procedure may be performed on soft tissues, hard tissues, or a combination thereof and may involve cutting, reshaping, rasping, contouring, repositioning, removing, repairing, grafting, or otherwise altering tissues to achieve a desired medical or functional outcome. A surgical procedure may be conducted through open surgery, minimally invasive techniques, robotic-assisted methods, or other medical approaches depending on the specific application.

In the context of osteotomy surgical procedures of the foot, a surgical procedure may include, but is not limited to, distal metatarsal osteotomy, proximal metatarsal osteotomy, diaphyseal osteotomy, Chevron osteotomy, Scarf osteotomy, Ludloff osteotomy, Akin osteotomy, Weil osteotomy, Cotton osteotomy, Evans osteotomy, Dwyer osteotomy, Lapidus procedure, closing wedge osteotomy, opening wedge osteotomy, and the like.

As used herein, an “interface,” “user interface,” or “engagement interface” refers to an area, a boundary, structure, or a place at which two separate and/or independent structures, members, apparatus, assemblies, components, and/or systems join, connect, are coupled, or meet and act on, or communicate, mechanically and/or electronically, with each other. In certain embodiments, “interface” may refer to a surface forming a common boundary of two bodies, spaces, structures, members, apparatus, assemblies, components, or phases. In certain embodiments, the term interface may be used with an adjective that identifies a type or function for the interface. For example, an engagement or coupling interface may refer to one or more structures that interact, connect, or couple to mechanically join or connect two separate structures, each connected to a side of the interface. In another example, a user interface may refer to one or more mechanical, electrical, or electromechanical structures that interact with or enable a user to provide user input, instructions, input signals, data, or data values and receive output, output data, or feedback.

The present disclosure discloses an apparatus, system and method for removing bone from a surface of one or more bones in a precise controlled manner. The present disclosure provides a rasp blade that connects to a powered oscillation handpiece. The rasp blade can provide a planar set of rasp features and provide visibility to enable a surgeon to make precise and accurate changes to the contour, size, and configuration of bone surfaces.

Advantageously, the present disclosure includes an apparatus with an angle feature that enables improved visibility for the surgeon using the apparatus. The angle feature enables a surgeon to use a rasp blade in a natural manner with high visibility and the assistance of gravity for very accurate and controlled contouring and fine tuning of bone surfaces. A surgeon can remove incremental amounts of bone in a controlled manner. Further, a surgeon can fine tune bone surfaces for example easily forming a flat planar surface to receive a fastener such as a bone staple and/or bone plate.

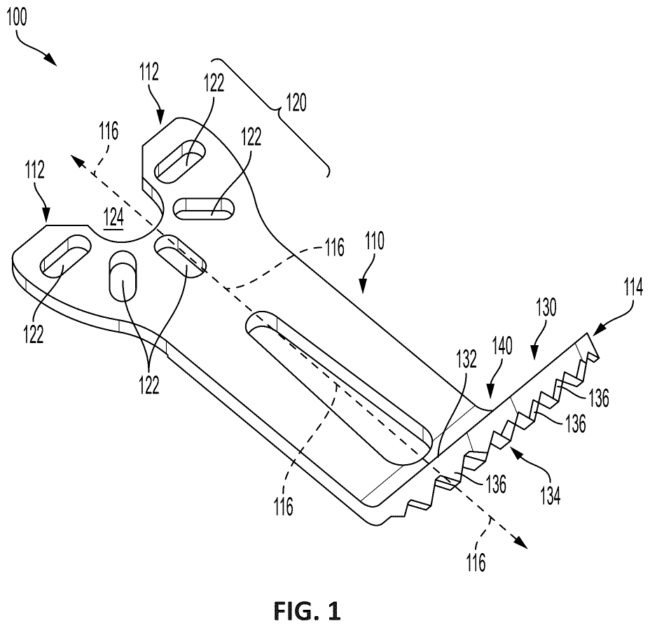

FIG. 1 is a perspective view of a rasp blade 100 for shaping a bone surface according to one embodiment. The rasp blade 100 may include, among other features and/or structures, a body 110, an oscillation mount 120, a foot 130, and an angle feature 140.

The body 110 provides a primary structural component of the rasp blade 100 and may define a generally planar, contoured, or shaped structure configured to support and connect other features of the rasp blade 100. The body 110 may serve as a mounting region for the oscillation mount 120 and may transition into the foot 130 through the angle feature 140. In another embodiment, the body 110 may connect or may be connectable to the oscillation mount 120 and/or the foot 130.

The geometry, thickness, and overall configuration of the body 110 may vary depending on factors such as the intended application, the type of oscillation handpiece used, and the desired interaction with the bone surface. In some embodiments, the body 110 may incorporate reinforcement structures, openings, surface treatments, and/or other design elements to enhance performance, stability, or material removal efficiency. The body 110 may be formed from various biocompatible or wear-resistant materials, including but not limited to metals, ceramics, polymers, or composite materials. In certain embodiments, the body 110 includes a proximal end 112, a distal end 114, and a longitudinal axis 116 that extends between the proximal end 112 and the distal end 114.

FIG. 1 illustrates one example configuration for an oscillation mount 120. The oscillation mount 120 may be substantially flat and may include a plurality of receiving slots 122 and a center clearance opening 124. The oscillation mount 120 may be clamped by a mount of a powered oscillation handpiece. A spring, not shown, may be disposed within the mount to provide a clamping force against the oscillation mount 120. One or more of the slots 122 may receive an engaging tine (not shown) of the powered oscillation handpiece. A wide variety of mount configurations are found in commercial use. While the inclusion of an oscillation mount 120 is necessary for the use of the presently disclosed rasp blade 100, the particular mount configuration of the oscillation mount 120 selected may not be critical to the disclosed rasp blade 100, so long as the mount configuration of the oscillation mount 120 is compatible with a mount of the powered oscillation handpiece with which the rasp blade 100 is used.

In the illustrated embodiment, the oscillation mount 120 is at the proximal end 112. The oscillation mount 120 is configured to securely couple the rasp blade 100 to a powered oscillation handpiece. Advantageously, the oscillation mount 120 is configured to enable use of the rasp blade 100 in common powered oscillation handpieces used in the industry. Thus, a special tool is not needed to use the rasp blade 100. Surgeon's can use one powered oscillation handpiece and readily change blades to move from a resection step to bone contouring using the rasp blade 100.

In one embodiment, the foot 130 is a structural component of the rasp blade 100 that extends from the body 110 and provides a primary working structure for engaging and modifying a bone surface. The foot 130 may be configured to facilitate controlled bone removal, shaping, or contouring through oscillations of the foot provided by a powered oscillation handpiece. The foot 130 may be dimensioned and shaped to provide a stable interface between the rasp blade 100 and one or more bone surfaces, enhancing precision and efficiency during use.

In certain embodiments, the foot 130 may include a superior side 132 and an inferior side 134. In one embodiment, the superior side 132 may transition from the body 110 and provide structural support for the foot 130, while the inferior side 134 may include a plurality of rasp features 136 that engage with a bone surface. In certain embodiments, the inferior side 134 is planar.

In the illustrated embodiment, the rasp features 136 may be configured to cut, abrade, shape, or smooth the bone surface, facilitating controlled material removal while minimizing unintended damage to surrounding tissue. The rasp features 136 may be arranged in a pattern optimized for efficient bone removal and may include variations in size, depth, spacing, or geometries to accommodate different surgical applications.

The foot 130 may be designed to enhance surgeon visibility and control, allowing for precise bone modification. In some embodiments, the foot 130 may be contoured, sized, and/or shaped and/or include features that help guide its interaction with the bone surface. In one embodiment, the foot 130 is a square or rectangular shape. In certain embodiments, the foot 130 has a length from the angle feature 140 to the distal end 114 of about 12.5 mm. In the illustrated embodiment, the foot 130 has a width that is the same or substantially the same as the width of the body 110. Additionally, the foot 130 may be configured to facilitate efficient evacuation of bone debris during operation, reducing clogging and maintaining consistent performance.

The geometry, thickness, and overall structure of the foot 130 may vary depending on factors such as an intended surgical procedure, a type of powered oscillation handpiece used, and one or more characteristics of a target material such as a target bone. The foot 130 may be integrally formed with the body 110 or may be a distinct structure affixed to the body 110. The foot 130 may be fabricated from various biocompatible or wear-resistant materials, including but not limited to stainless steel, titanium, ceramics, polymers, or composite materials.

Advantageously, the foot 130 provides a stable and effective rasping surface that can be used in conjunction with powered oscillation handpieces to facilitate bone reshaping, contouring, or preparation for surgical procedures, such as implant placement. The configuration of the foot 130, such as its angle relative to the longitudinal axis 116 enables the rasp blade 100 to be used in a variety of surgical applications, allowing surgeons to efficiently modify bone surfaces without requiring specialized tools or hand instruments.

The angle feature 140 provides a structural connection between the body 110 and the foot 130. In the illustrated embodiment, the angle feature 140 orients the foot 130 at an acute angle relative to the longitudinal axis 116 of the body 110. This angular relationship is designed to optimize the engagement of the rasp features 136 with the bone surface, allowing for effective and controlled material removal. The angle feature 140 in certain embodiments may be configured to maintain structural integrity while transmitting oscillatory motion efficiently from the body 110 to the foot 130.

In some embodiments, the angle feature 140 may be formed as a continuous transition between the body 110 and the foot 130, such as an arcuate or curved segment. This configuration may provide smooth force distribution and minimize stress concentrations, enhancing the durability and reliability of the rasp blade 100 during use. Alternatively, or in addition, the angle feature 140 may be implemented as a distinct structural element, such as a linear or stepped transition, which may provide defined articulation between the body 110 and the foot 130, allowing for precise positioning and/or orienting of the rasp features 136.

Various alternative embodiments of the angle feature 140 may include flexible or adjustable configurations, enabling the foot 130 to be positioned at different angles relative to the body 110 to accommodate varying anatomical structures or surgical preferences. In some implementations, the angle feature 140 may include a locking or pivoting mechanism that allows a surgeon to modify the angular orientation of the foot 130 before and/or during an operation. Additionally, in certain embodiments, the angle feature 140 may incorporate reinforcement structures, such as ribs or fillets, to improve strength and resistance to mechanical stresses during oscillatory motion.

Advantageously, the design and configuration of the angle feature 140 provides an enhanced cutting geometry that allows for more efficient bone removal and improved surgical precision. The acute angle orientation facilitated by the angle feature 140 ensures that the rasp features 136 are optimally positioned for engagement with a bone surface while maintaining stability and control. This allows the rasp blade 100 to be effectively utilized across various surgical applications, providing a versatile and effective tool for bone shaping and contouring.

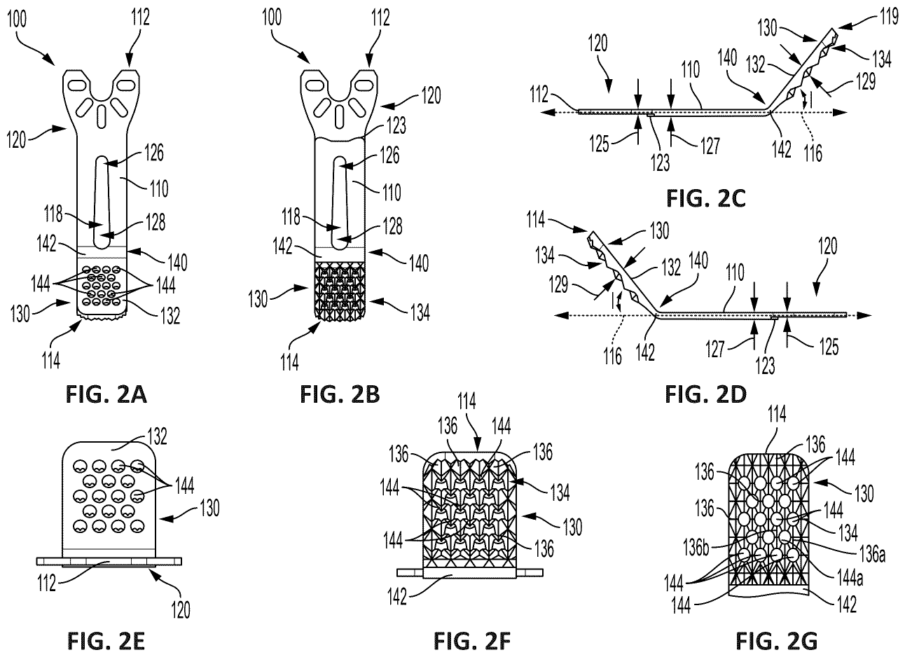

FIGS. 2A-2G illustrate views of the rasp blade of FIG. 1, according to one embodiment. Specifically, FIG. 2A illustrates a superior side view of the rasp blade 100. FIG. 2B illustrates an inferior side view of the rasp blade 100. FIG. 2C illustrates a left-side view of the rasp blade 100. FIG. 2D illustrates a right-side view of the rasp blade 100. FIG. 2E illustrates a proximal end view of the rasp blade 100. FIG. 2F illustrates a distal end view of the rasp blade 100. And FIG. 2G illustrates an inferior side view of the foot 130 of the rasp blade 100.

FIGS. 2A-2G illustrate various views of the rasp blade 100, highlighting structural features that contribute to its functionality and advantages in shaping a bone surface. These figures provide further details regarding the configuration of the foot 130, the angle feature 140, and the oscillation mount 120.

FIG. 2A is a top or superior side view of the rasp blade 100, illustrating the superior side 132 of the foot 130. In this embodiment, the superior side 132 is planar, providing a stable and structured surface that enhances the structural integrity of the rasp blade 100.

In the illustrated embodiment, the foot 130 includes a plurality of openings 144 that extend from the superior side 132 to the inferior side 134. These openings 144 are configured to facilitate the evacuation of bone debris during operation, reducing clogging and improving cutting efficiency. The openings 144 may vary in shape, size, and distribution to optimize debris removal while maintaining the structural strength of the foot 130. In certain embodiments, the openings 144 have a radius of between about 1 mm to about 3 mm. In some embodiments, the openings 144 may be arranged in a pattern that directs debris away from the working surface of the rasp blade 100 to prevent interference with bone modification.

FIG. 2B is a bottom or inferior side view of the rasp blade 100, showing the body 110, the oscillation mount 120, foot 130 (including the rasp features rasp features 136), and the angle feature 140. In one embodiment, the body 110 is a single unitary piece that includes an opening. The opening may have various shapes and/or sizes. In the illustrated embodiment, the opening is a shaped opening 118. The shaped opening 118 may be shaped like a slot with a proximal end 126 and a distal end distal end 128. In the illustrated embodiment, the distal end 128 has a larger diameter than the proximal end 126. In certain embodiments, the shaped opening 118 is sized and/or configured to optimize the mass and/or weight of the rasp blade 100.

In another embodiment, the body 110 includes two or more arms/legs that connect the oscillation mount 120 and the angle feature 140. The arms/legs may be planar and may provide structural integrity for the body 110.

In one embodiment, the angle feature 140 includes an inclination curve 142 that connects the body 110 and the foot 130. The inclination curve 142 facilitates a smooth transition between the body 110 and the foot 130, optimizing engagement with the bone surface while maintaining efficient transmission of oscillatory motion. The inclination curve 142 may be configured to improve the ergonomic handling and functional performance of the rasp blade 100, ensuring controlled material removal. The specific curvature of the inclination curve 142 may be selected based on surgical applications, optimizing accessibility to targeted bone structures.

FIG. 2C provides a left-side view of the rasp blade 100, illustrating the relative thicknesses of certain structural elements. The oscillation mount 120 has an oscillation mount thickness 125, the body 110 has a body thickness 127, and the foot 130 has a foot thickness 129. In certain embodiments, the oscillation mount thickness 125 is about 0.55 mm and the body thickness 127 is about 0.7 mm and the foot thickness 129 is about 1.5 mm.

In the illustrated embodiment, the body thickness 127 is greater than the oscillation mount thickness 125. In certain embodiments, this difference in thickness may be evidenced by a lip 123 between the body 110 and the oscillation mount oscillation mount 120.

This design enhances the structural rigidity and durability of the rasp blade 100 while allowing the oscillation mount 120 to securely engage with a conventional powered oscillation handpiece. The difference in thickness ensures that the body 110 can withstand mechanical stresses of oscillating during operation while maintaining compatibility with common oscillation handpieces. In some embodiments, the variation in thickness between the oscillation mount 120 and the body 110 may contribute to improved handling, vibration control, and/or force transmission. As discussed later herein the difference in thickness can be configured to manage the mass moment of inertia of the rasp blade 100 to obtain a desired mass moment of inertia.

FIG. 2C also illustrates the angle feature 140 in relation to the body 110 and the foot 130. The angle feature 140 positions and orients the foot 130 relative to the body 110. In one embodiment, this can be done by connecting the body 110 to the foot 130 at an acute angle relative to the longitudinal axis longitudinal axis 116. In the illustrated embodiment, an acute angle is embodied as an inclination angle I relative to the longitudinal axis 116 of the body 110. In certain embodiments, the inclination angle I is measured in degrees and can range from about 30 degrees to about 60 degrees. In one embodiment, the inclination angle I is 45 degrees.

FIG. 2D illustrates a right-side view of the rasp blade 100. FIG. 2D shows that the rasp blade rasp blade 100, in one embodiment, has a symmetrical design, meaning that its right-side configuration mirrors its left-side configuration. This symmetry can ensure consistent functionality and uniform performance regardless of the orientation. Such a design may enhance balance, ease of use, and manufacturing efficiency.

FIGS. 2C and 2D also illustrate an example of an angle feature 140 implemented using an inclination curve 142. These FIGs. show a clear view of an implementation of a rasp blade 100 with a planar superior side 132. In the illustrated embodiment, the planar superior side 132 is smooth. Similarly, the inferior side 134 includes rasp features 136 that, in one embodiment, are configured to form a planar cut face.

FIG. 2E illustrates a proximal end view of the rasp blade 100. The superior side 132 of the foot 130 is illustrated as well as the part of the openings 144 that extend through the superior side superior side 132. An embodiment of a smooth planar superior side 132 is also shown. This FIG. 2E shows an example of a planar configuration of the superior side 132 and the plurality of openings 144.

FIG. 2F illustrates a distal end view of the rasp blade 100. The inferior side 134 and plurality of rasp features 136 are illustrated. In the illustrated embodiment, the rasp features 136 are organized into a pattern on the inferior side 134 of the foot 130. Also illustrated is a plurality of openings 144 distributed between rasp features 136.

And FIG. 2G illustrates an inferior side view of the foot 130 of the rasp blade 100. This is a plan view of the inferior side 134. The openings 144 in this embodiment are circular in shape and uniform. Of course, in other embodiments the openings 144 can have a variety of sizes and/or shapes and can be distributed according to different patterns. In one embodiment, at least one opening 144 of the plurality of openings 144 is disposed between at least two rasp features 136. See for example, opening 144a disposed between rasp feature 136a and rasp feature 136b.

Collectively, FIGS. 2A-2G highlight certain features of the rasp blade 100 that contribute to its improved functionality in bone shaping and contouring. The inferior side 134 of the foot 130 provides a stable surface, while the plurality of openings 144 facilitate efficient bone debris evacuation. The angle feature 140, including the inclination curve 142, ensures an optimized connection between the body 110 and the foot 130, enhancing the tool's cutting efficiency. Additionally, the difference in thickness between the oscillation mount 120 and the body 110 provides enhanced structural integrity while ensuring compatibility with powered oscillation handpieces.

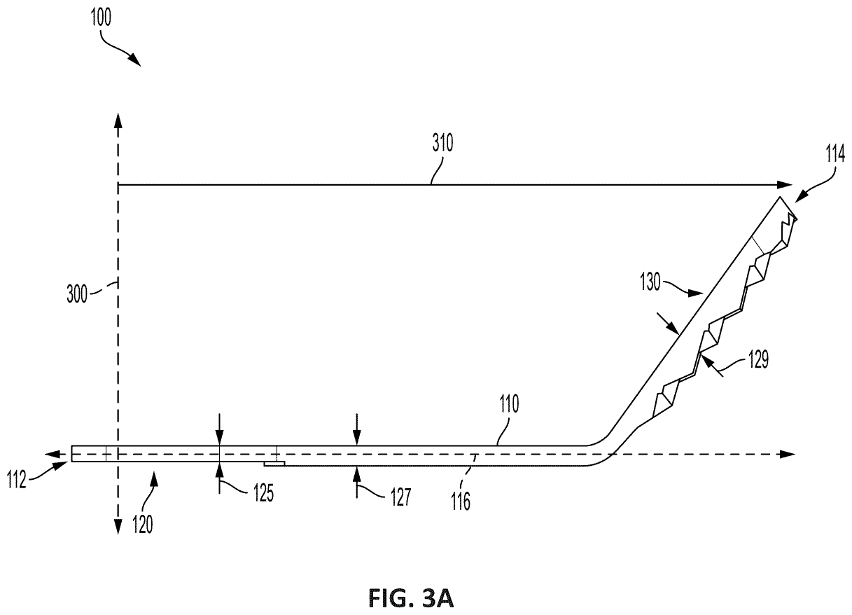

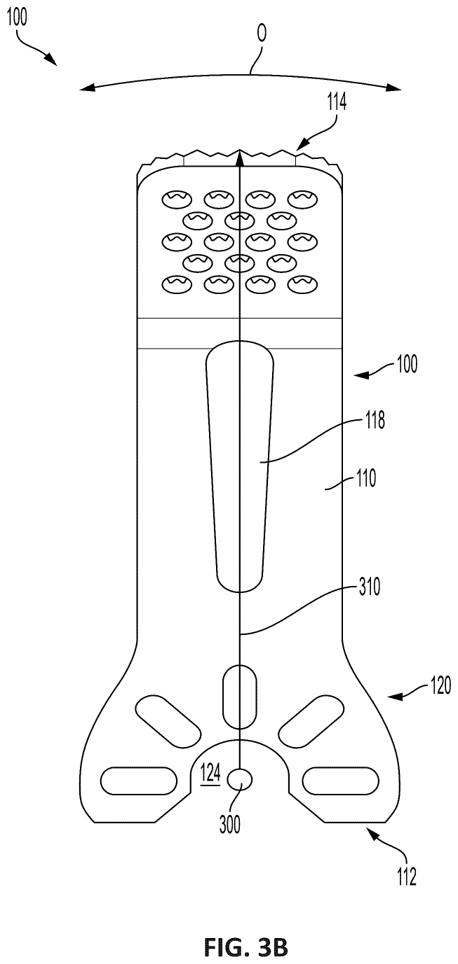

FIG. 3A illustrates a side view a rasp blade 100 according to one embodiment. FIG. 3A illustrates a close-up view of the rasp blade 100 and shows differences in the oscillation mount thickness 125, body thickness 127, and foot thickness 129. The longitudinal axis 116 and a pivot axis 300 is illustrated.

The pivot axis 300 is the axis about which the rasp blade 100 oscillates when operated by a powered oscillation handpiece. The pivot axis 300 is perpendicular to the longitudinal axis 116. In one embodiment, the pivot axis 300 passes through the center of the center clearance opening 124 of the oscillation mount 120.

One challenge addressed by embodiments of the present disclosure is ensuring that the rasp blade 100 remains compatible with conventional powered oscillation handpieces while maintaining structural integrity during operation. Specifically, the rasp blade 100 is designed to prevent breakage of the oscillation mount 120 and to securely engage with the powered oscillation handpiece under typical operating conditions.

One factor in overcoming this challenge is to configure the rasp blade 100 to achieve a desired mass moment of inertia (aka moment). To achieve a mass moment of inertia within a desired operational range, the design of the rasp blade 100 is configured to optimize resistance to angular acceleration about the pivot axis 300 of rotation. Various structural elements, including the oscillation mount 120, body 110, and foot 130, are dimensioned and positioned to achieve this balance. The oscillation mount 120, body 110, and foot 130 are configured to achieve the desired mass moment of inertia. The mass of each component is designed to achieve functional objectives while maintaining a mass moment of inertia within an operational range of about 725 gmm² to about 750 gmm². In certain embodiments, the rasp blade 100 is configured to have a mass moment of inertia of approximately 738 g*mm², though variations may be implemented based on application-specific requirements

Another factor in managing the operational dynamics of the rasp blade 100 is the moment arm 310. The moment arm 310 of the rasp blade 100 is configured to provide optimal performance and rasping functionality while operating within the mechanical tolerances of the powered oscillation handpiece. The moment arm 310 is measured from the pivot axis 300 to the distal end 114 where the foot 130 ends and parallel to the longitudinal axis 116. In various embodiments, the moment arm 310 is configured to fall within an operational range of about 25 mm to about 40 mm. In one example embodiment, the moment arm 310 is approximately 36 mm, though variations may be made to optimize performance for specific powered oscillation handpieces.

FIG. 3B illustrates a superior side view (or plan view) of the rasp blade 100, as shown in FIG. 3A. This view further depicts the pivot axis 300, moment arm 310, and the oscillation movement of the rasp blade 100 when engaged with a powered oscillation handpiece, as indicated by arrow O.

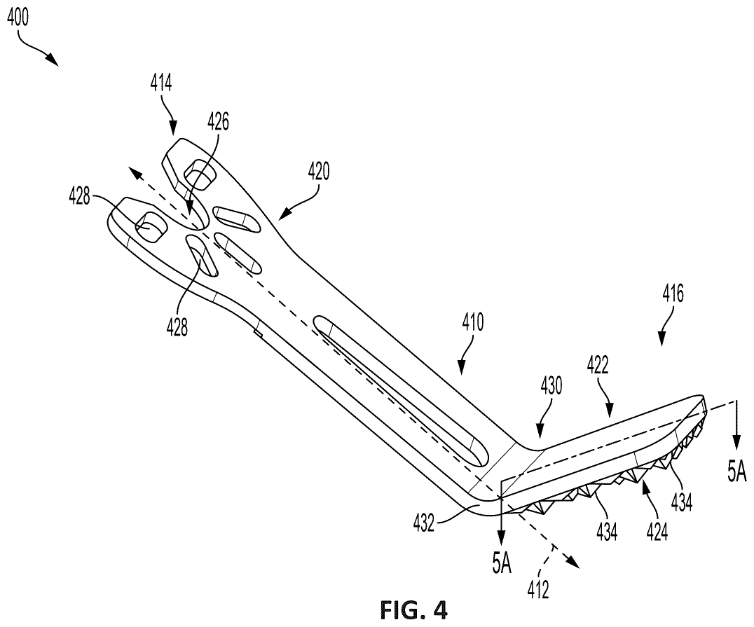

FIG. 4 is a perspective view of a rasp blade according to one embodiment. FIG. 4 illustrates an embodiment of a surface contouring blade 400, which is configured for contouring a bone surface. The surface contouring blade 400 includes a body 410 extending along a longitudinal axis 412, an oscillation mount 420 at the proximal end 414, and a distal end 416. The distal end 416 includes a superior side 422, an inferior side 424, and an inclination feature 430.

The body 410 serves as a primary structural component of the surface contouring blade 400, extending along the longitudinal axis 412 from the proximal end 414 to the distal end 416. The body 410 may be formed from a biocompatible metal, such as stainless steel or titanium, and/or from a high-strength composite material. The body 410 is dimensioned to provide sufficient rigidity to support oscillatory motion while maintaining flexibility to accommodate rasping, shaping, and/or contouring forces exerted during use.

In certain embodiments, the body 410 may have varying thicknesses along its length to optimize strength, compatibility, and maneuverability. In certain embodiments, the body 410 may include structural reinforcements or surface coatings to enhance wear resistance. In one embodiment, the body 410 is configured to integrate with the oscillation mount 420 towards the proximal end 414 and transition into the inclination feature 430 towards the distal end 416.

The oscillation mount 420 is positioned at the proximal end 414 of the surface contouring blade 400 and is configured to couple the blade to a powered oscillation handpiece. The oscillation mount 420 may include a center clearance opening 426, slots 428, and/or other engagement features designed to interface securely with the drive mechanism of the handpiece.

The oscillation mount 420 can be manufactured from the same material as the body 410 or from a reinforced alloy to withstand repeated oscillation cycles. The oscillation mount 420 may be designed to accommodate various handpiece models by including universal attachment features and/or interchangeable connectors. The configuration of the oscillation mount 420 ensures that the surface contouring blade 400 can operate efficiently without excessive vibration and a mitigated risk of detachment.

The distal end 416 of the surface contouring blade 400 can include several functional elements, including a superior side 422, an inferior side 424, an inclination feature 430, and a plurality of teeth 434. The distal end 416 is configured to facilitate controlled bone contouring by engaging the inferior side 424 against the bone surface. In certain embodiments, the distal end 416 is planar. In the illustrated embodiment, the distal end 416 may not include openings between the plurality of teeth 434. Alternatively, or in addition, the distal end 416 can include one or more teeth 434.

The superior side 422 of the distal end 416 can extend along an upper surface of the surface contouring blade 400. In the illustrated embodiment, the superior side 422 is planar and smooth. In some embodiments, the superior side 422 may include structural reinforcements or surface treatments to enhance durability. The superior side 422 may have a contoured or beveled design to reduce resistance when maneuvering the blade and/or to reduce weight or mass of the surface contouring blade 400.

In certain embodiments, the superior side 422 may include depth markers, etching, or coating variations that assist in guiding the user during a rasping and/or contouring process. These features may help maintain precision when performing controlled bone reduction.

The inferior side 424 of the distal end distal end 416, in the illustrated embodiment, is planar. Of course, the inferior side 424 can have other geometric shapes and/or configurations including square, rectangular, oval, ellipse, curved, arcuate, concave, convex, and the like.

The inferior side 424 of the distal end 416 includes a plurality of teeth 434, which are configured to engage with and remove bone to shape and/or contour a bone surface. The plurality of teeth 434 may be formed as a continuous serrated edge, a segmented pattern, or an array of individually shaped cutting elements or cutting/rasping/abrading teeth.

The plurality of teeth 434 may have various profiles, such as triangular, chisel-shaped, rounded, or the like, depending on the intended application. The spacing and depth of the plurality of teeth 434 can be optimized to provide efficient material removal while minimizing trauma to surrounding tissue. In some embodiments, the plurality of teeth 434 may include a coating or surface treatment to improve longevity and/or reduce friction.

The inferior side 424 ensures that the plurality of teeth 434 remain in optimal alignment and position relative to the bone surface, allowing for consistent and predictable contouring.

Advantageously, the distal end 416 includes an inclination feature 430 that optimally positions and/or orients the distal end 416. In one embodiment, the inclination feature 430 tilts the distal end 416 of the body 410 at an acute angle relative to the longitudinal axis longitudinal axis 412. The inclination feature 430 is configured to position the distal end 416 of the surface contouring blade 400 at an acute angle measured relative to the longitudinal axis 412. This acute angle facilitates controlled engagement between the plurality of teeth 434 and the bone surface, allowing for precise contouring while reducing the need for excessive force. In one embodiment, the acute angle is 45 degrees and the surface contouring blade 400 extends coincident with the longitudinal axis of the powered oscillation handpiece.

This 45-degree angle enables a surgeon to hold the powered oscillation handpiece and thus surface contouring blade 400 in a natural and normal position for use when contouring a bone surface. Advantageously, where the surgeon has a direct approach to the bone, with the bone and operating field directly in front of and at arms height of the surgeon use of the powered oscillation handpiece and surface contouring blade 400 is very natural and normal. Minimal strain and stress is placed on the surgeon positioning the surface contouring blade 400. Furthermore, the inclination feature 430 positions the plurality of teeth 434 at a common and understood angle relative to the surface contouring blade 400. A surgeon can move the plurality of teeth 434 onto a bone surface by simply extending the surface contouring blade 400 directly forward and know that they will be engaging the bone surface at 45 degrees. Alternatively, or in addition, when the surgeon wants to contour a bone surface to form a planar surface that is parallel to the surface contouring blade surface contouring blade 400, the surgeon only needs to tilt the powered oscillation handpiece forward 45 degrees to form the desired new contoured bone surface.

The inclination feature 430 may be designed with a fixed or variable angle, depending on the embodiment. In some cases, the inclination feature 430 may include reinforcement structures to maintain its angled position under load. The angle of the inclination feature 430 can be optimized based on surgical requirements, with possible angles ranging from 10 degrees to 60 degrees in different embodiments. Those of skill in the art will appreciate that the inclination feature 430 may orient the distal end 416 in a positive inclination or a negative inclination with respect to the longitudinal axis 412.

In some embodiments, the inclination feature 430 includes an inclination curve 432, which may bias the distal end 416 to return to an acute angle when the plurality of teeth 434 engages a bone surface. In one embodiment, the inclination feature 430 includes an inclination curve 432 that connects the distal end 416 to the body 410. The inclination curve 432 may provide a spring-like biasing effect, allowing the distal end 416 to return to its acute angle after being pressed against a bone surface. Specifically, in one embodiment, the inclination curve 432 biases the distal end 416 to return to the acute angle in response to pressing the plurality of teeth 434 against a bone surface. The inclination curve 432 may be formed as a continuous arc, a stepped transition, or a flexible joint, depending on the embodiment. The geometry of the inclination curve 432 may be tailored to balance flexibility and stability, ensuring that the plurality of teeth 434 maintain effective contact with the bone surface during oscillation.

In some embodiments, the inclination curve 432 is manufactured using a flexible or elastically deformable material, such as a specially treated metal alloy, to enhance its biasing properties. Alternatively, or in addition, structural reinforcements may be incorporated to control the degree of flexibility and resistance to deformation.

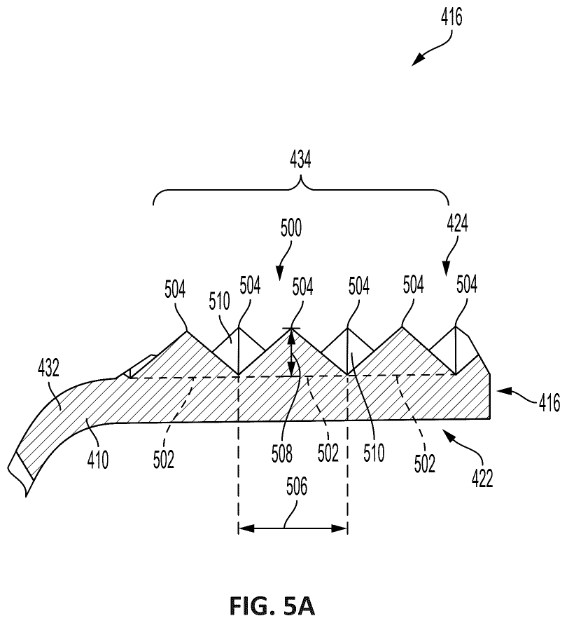

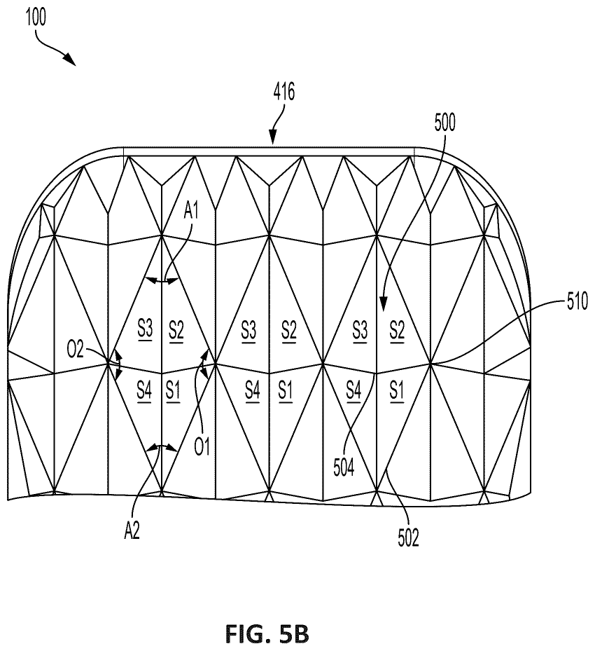

FIG. 5A is a cross-section view of the surface contouring blade 400 of FIG. 4 taken along lines 5-5 according to one embodiment. FIG. 5A illustrates the distal end distal end 416, with the superior side 422 on the bottom side of the illustration, the inferior side 424 is on the top side of the illustration, and the inclination curve 432. A cross-section view of a plurality of teeth 434. Each tooth 500 includes a base 502 and a tip 504. In the illustrated embodiment, the base 502 is connected to or integrated with the body 410. The tip 504 is the part of the tooth 500 that engages with the target material (i.e., bone). In certain embodiments, the tip 504 is embodied as an abrading feature.

Those of skill in the art will appreciate that a tooth 500 may vary in size, shape, configuration, and spacing depending on the specific application and desired cutting or abrading characteristics. In some embodiments, a tooth 500 may have a uniform size and shape, such as triangular, chisel-shaped, rounded, conical, or the like, to provide consistent engagement with the target material. In other embodiments, one or more teeth 500 may have varying height, width, or angle to facilitate differential material removal, enhance efficiency, or reduce resistance during use. The spacing between adjacent teeth may also vary, with closely spaced teeth providing finer abrasion and more gradual contouring, while widely spaced teeth may allow for more aggressive material removal.

The positioning and orientation of the teeth 500 can further be adapted to suit different applications. For example, in some embodiments, teeth 500 may be arranged in linear rows, staggered patterns, arcuate formations, helical configurations, or the like to optimize cutting efficiency and reduce clogging during use. In certain configurations, the base 502 of the tooth may be angled or curved relative to the body 410 to improve material removal dynamics or accommodate oscillatory movement. Additionally, the tip 504 may include sharp edges, micro-serrations, or surface texturing to enhance cutting or abrading performance, depending on whether the instrument is designed for aggressive cutting, smoothing, or controlled reshaping.

In some embodiments, the tooth 500 may be coated, reinforced, or treated with materials such as diamond grit, tungsten carbide, or ceramic coatings to improve durability, enhance wear resistance, or modify frictional properties. Alternatively, a tooth 500 may be integrally formed from the same material as the body 410, such as through precision machining, laser structuring, or additive manufacturing processes, to achieve a seamless and structurally robust design. Variations in these features allow the surface contouring blade 400 to be tailored for a wide range of applications, from fine surface smoothing to aggressive material removal in surgical or orthopedic procedures.

The plurality of teeth 434 may include both full teeth 500 and partial teeth 500. Generally, a tooth 500 has a width 506 measured from one side of the base 502 to the other side of the base 502. A tooth 500 also has a height 508 measured from the base 502 to the tip 504. Space between teeth 500 is referred to as a gullet 510. The size of the gullet 510 is one example of and/or representation of a tooth separation distance. A tooth separation distance is a measure of a distance between teeth 500 or a distance between parts of two teeth 500 (full or partial). For partial teeth, the width 506 and/or height 508 may be shortened due to the application and/or due to the position of the partial tooth 500 relative to an edge of the distal end 416.

Referring now to FIG. 5B, FIG. 5B is close-up view of teeth according to one embodiment. In the illustrated embodiment, each tooth 500 has a uniform and/or similar design, shape, size, and/or configuration. Of course, these can be varied in different embodiments for different applications. Each tooth 500 has base 502 (See FIG. 5A) and a tip 504 that serves as an abrading feature. In the illustrated embodiment, the base 502 is connected to the tip 504 by four sides, S1, S2, S3, S4. The first side S1 extends from the base 502 to the tip 504 and is adjacent to the second side S2. The second side S2 extends from the base 502 to the tip 504 and is adjacent to the third side S3. The third side S3 extends from the base 502 to the tip 504 and is adjacent to the fourth side S4. The fourth side S4 extends from the base 502 to the tip 504 and is adjacent to the third side S3 and the first side S1. Each of the four sides (S1, S2, S3, and S4) converge at the tip 504.

FIG. 5B also illustrates how adjacent sides meet with each other to form a tooth 500. In one example, the first side S1 meets the second side S2 at the base at an obtuse angle O1. The second side S2 meets the third side S3 at the base at an acute angle A1. The third side S3 meets the fourth side S4 at the base at an obtuse angle O2. And, the fourth side S4 meets the first side S1 at the base at an acute angle A2. These angles O1, O2, A1, A2 for the sides S1, S2, S3, and S4 form a pyramid shape the comes to a point at the tip 504. Each S1, S2, S3, and S4 can serve as a cut face for the surface contouring blade 400.

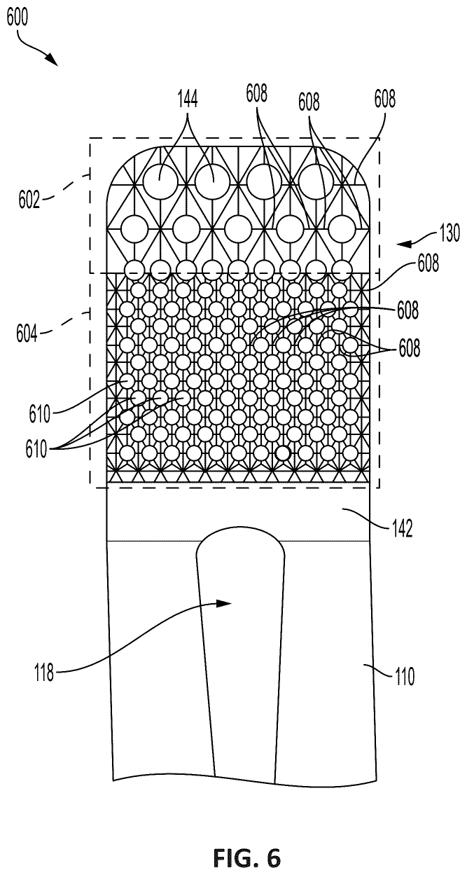

FIG. 6 is an inferior side view of an alternative embodiment for a rasp blade 600 according to one embodiment. The rasp blade 600 may have many structures, features, and functions, operations, and configuration similar or identical to those of the rasp blade 100 described in relation to FIGS. 1-3B, like parts are identified with the same reference numerals. Accordingly, the rasp blade 600 may include a body 110, a shaped opening 118, an inclination curve 142, and a foot 130 having an inferior side 134.

The inferior side 134 includes rasp features 136, however in this embodiment the rasp features 136 are configured differently than the rasp blade 100 embodiment. In the illustrated embodiment, the rasp features 136 are configured to provide a coarse rasp feature 602 and a fine rasp feature 604. Those of skill in the art will appreciate that the coarse rasp feature 602 and/or fine rasp feature 604 can be implemented using a variety of different designs, rasp features 136, rasp feature 136 configurations, rasp feature 136 patterns, and the like. Also, the position and/or organization of the coarse rasp feature 602 and/or fine rasp feature 604 can be varied in different embodiments. In the illustrated embodiment, the coarse rasp feature 602 is closer to the distal end 114 and the fine rasp feature 604 is closer to the inclination curve 142. Of course, these positions can be reversed in other embodiments.

In the illustrated example embodiment, the coarse rasp feature 602 and fine rasp feature 604 are implemented using two sets of rasp features 136. For the coarse rasp feature 602, the rasp features 136 may be sized, shaped, configured and/or positioned in a similar manner to those of the embodiments shown in FIGS. 1-3B. For the fine rasp feature 604, the fine rasp feature 604 may include rasp features 608 that are closer together, have a shorter height, have fewer sides, are positioned in a different pattern. Alternatively, or in addition, for the fine rasp feature 604, the fine rasp feature 604 may include openings 610 like openings 144, but the openings 610 may have a different shape and/or a smaller diameter. Alternatively, or in addition, the fine rasp feature 604 rasp features 608 may includes sides that have a cut face at a different angle than rasp features 136 of a coarse rasp feature 602. In at least one embodiment, the coarse rasp feature 602 includes a set of teeth (e.g., rasp features 136) with at least one of a tooth height and a tooth separation distance that is greater than a corresponding at least one of a tooth height and a tooth separation distance for a set of teeth (e.g., rasp features 608) of the fine rasp feature 604.

The coarse rasp feature 602 and the fine rasp feature 604 may provide a greater level of control for a surgeon regarding how aggressively bone material is removed. As rasping or bone removal by abrasion is a process that is impossible or extremely difficult to reverse, a surgeon may be inclined to start bone removal with a fine rasp feature 604 and based on those results determine whether additional bone removal or contouring is needed using the fine rasp feature 604 or the coarse rasp feature 602. Alternatively, or in addition, a surgeon may start with the coarse rasp feature 602 and then transition to the fine rasp feature 604 as the bone removal begins to meet their desired outcome. Advantageously, having a single rasp blade 600 with both a coarse rasp feature 602 and a fine rasp feature 604 provides this functionality without changing the blade of the powered oscillation handpiece.

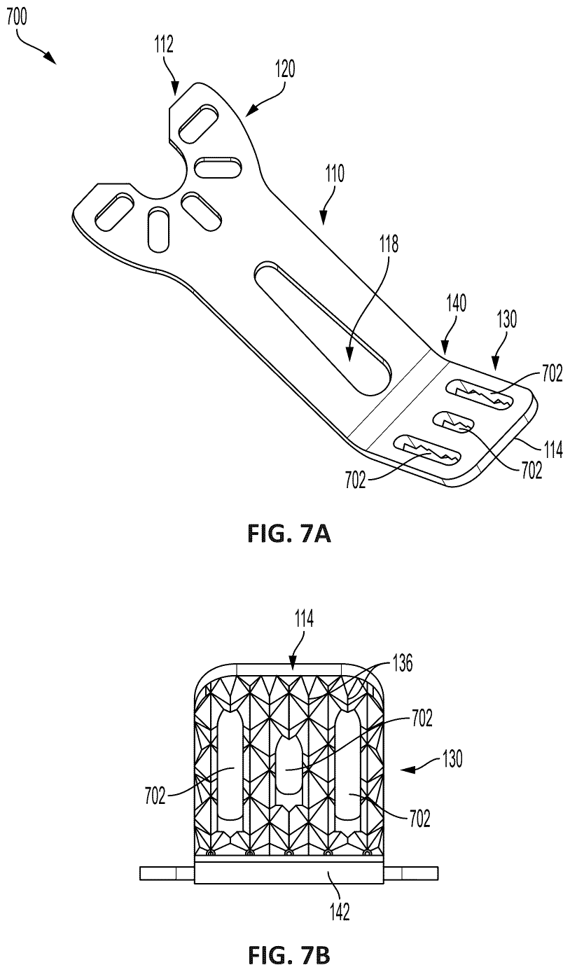

FIGS. 7A and 7B is a perspective view and a distal end view, respectively of an alternative embodiment for a rasp blade 700 according to one embodiment. The rasp blade 700 may have many structures, features, and functions, operations, and configuration similar or identical to those of the rasp blade 100 described in relation to FIGS. 1-3B. Like parts are identified with the same reference numerals. Accordingly, the rasp blade 700 may include a body 110, a proximal end 112, distal end 114, a shaped opening 118, a foot 130, angle feature 140, an inclination curve 142, and rasp features 136.

In the illustrated embodiment, one difference between the rasp blade 700 and the rasp blade 100 is that the rasp blade 700 includes a different configuration of openings 144. Rather than evenly spaced circular openings, the rasp blade 700 includes one or more slots 702. The slots 702 can be positioned, sized, and/or shaped to optimize evacuation of bone debris during a bone removal procedure using the rasp blade 700.

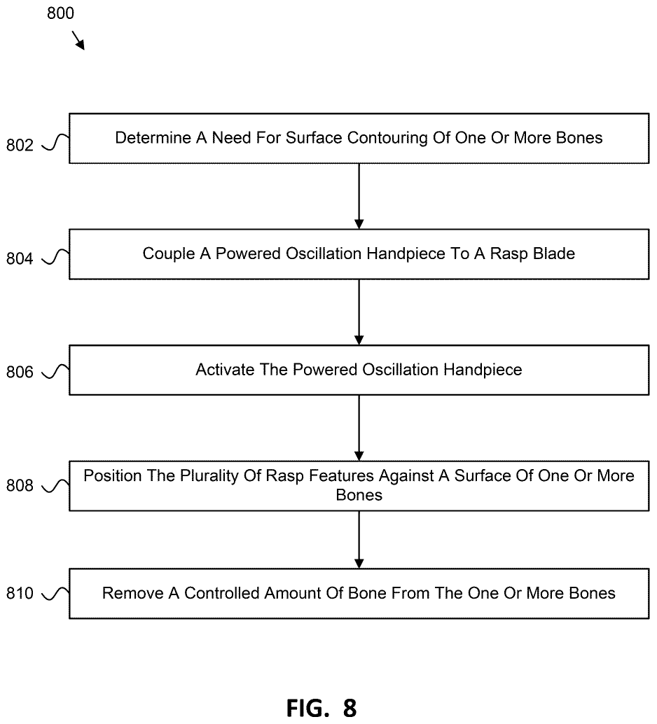

FIG. 8 is a flowchart diagram depicting a method 800 or process 800 for remediating a bone condition present in a patient's foot, according to one embodiment. In some implementations, one or more process blocks of FIG. 8 may be performed in whole or in part by a surgeon, an apparatus, device and/or system of the present disclosure. As shown in FIG. 8, process 800 may include determining a need for contouring one or more bones of a patient in a surgical procedure (block 802). For example, a surgical technique or a surgeon may determine a need for surface contouring, as described above.

As also shown in FIG. 8, process 800 may include coupling a powered oscillation handpiece to a rasp blade (block 804). The rasp blade may include a body having a proximal end, a distal end, and longitudinal axis that extends from the proximal end to the distal end. The rasp blade may further include an oscillation mount at the proximal end. The oscillation mount may be configured to couple the rasp blade to a powered oscillation handpiece.

The rasp blade may include a foot that includes a superior side and a planar inferior side that includes a plurality of rasp features. The rasp blade may also include an angle feature that connects the foot and the body at an acute angle relative to the longitudinal axis of the body.

As further shown in FIG. 8, process 800 may include activating the powered oscillation handpiece (block 806). Next, process 800 may include positioning the plurality of rasp features against a surface of one or more bones of a patient (block 808). Next, process 800 may include removing a controlled amount of bone from the one or more bones (block 810).

In certain embodiments, the process 800 may also include exchanging a first rasp blade coupled to the powered oscillation handpiece with a second rasp blade. In such an embodiment, the first rasp blade may include a coarse rasp feature and the second rasp blade may include a fine rasp feature.

Although FIG. 8 shows example blocks of process 800, in some implementations, process 800 may include additional blocks, fewer blocks, different blocks, or differently arranged blocks than those depicted in FIG. 8. Additionally, or alternatively, two or more of the blocks of process 800 may be performed in parallel.

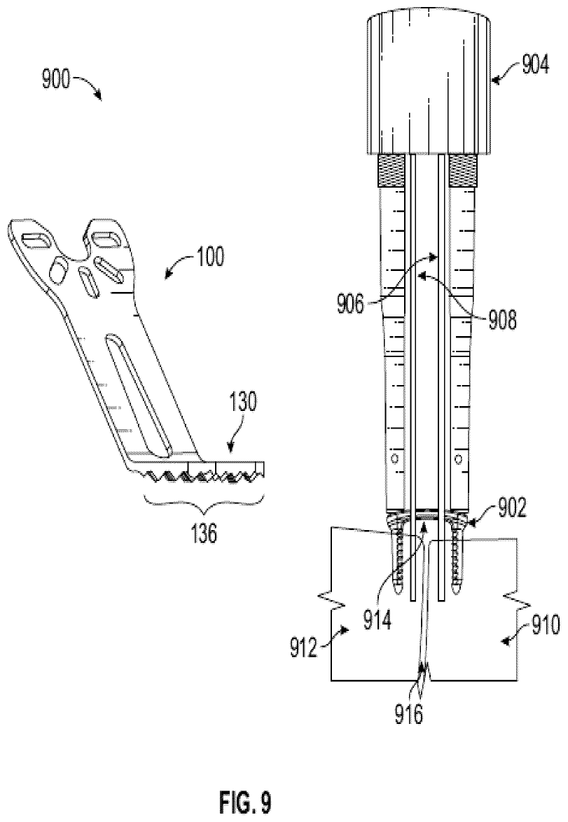

FIG. 9 illustrates an exemplary system 900 configured to provide fixation to two bones using a rasp blade 100 according to one embodiment. The system 900 includes a rasp blade 100 and a staple 902.

FIG. 9 illustrates a stage of a surgical procedure using the system 900, according to one embodiment. A surgeon has positioned and fitted (aka trialed) the placement of a staple 902. In particular, a user is advancing an inserter 904 along a first wire 906 and a second wire 908 to position the staple 902 in contact with a first bone 910 and a second bone 912. A bridge 914 of the staple 902 bridges across a space 916 between the first bone 910 and the second bone 912.

The advancement of the inserter 904 can include positioning a first leg in a first implant hole in the first bone 910 and positioning a second leg in a second implant hole in the second bone 912. Such advancing the inserter 904 can include contacting the first bone 910 and the second bone 912 with a bottom surface 914 of the staple 902. In one specific example, contacting the first bone 910 and the second bone 912 with the bottom surface 914 of the staple 902 can include contacting the first bone 910 generally flush with the bottom surface 914 and contacting the second bone 912 generally flush with the bottom surface 914.

After fitting the staple 902 with respect to the holes and/or the surfaces of the first bone 910 and the second bone 912, a user may determine that bone removal is needed to provide an optimally shaped bone surface of the first bone 910 and/or second bone 912 that will contact the bottom surface 914 of the staple 902 when the staple 902 is deployed. Specifically, a user may desire an even and/or planar surface of bone where the staple 902 contacts the first bone 910 and/or second bone 912. To achieve this, a user may use one embodiment of a rasp blade described in the present disclosure. Such embodiments are useful in removing undesired bone such as osteophytes which may be done to ensure a flush fit of fixation implants. Advantageously, the rasp blade enables the user to remove a controlled amount of bone and/or shape one or more bone surfaces to get a desired shape (i.e., a planar surface).

Consequently, a user may temporarily remove the inserter 904 and staple 902 for bone removal. The user may optionally remove or leave in place the first wire 906 and second wire 908. Next, a user may couple a rasp blade 100 to a powered oscillation handpiece and activate the powered oscillation handpiece. The user may then position the oscillating foot 130 and rasp features 136 against the bone surfaces of the first bone 910 and the second bone 912 and remove bone in preparation for deployment of the staple 902.

Advantageously, the rasp features 136 provide a planar cut face that a user can position with precision to form a planar surface on one or more of the first bone 910 and the second bone 912. In addition, the control provided by the rasp blade 100 can enable a user to remove bone around the first wire 906 and/or second wire 908 without removing them. The user can repeatedly remove just a desired amount of bone to form a planar surface of the first bone 910 and second bone 912 to contact the bottom surface 914 of the staple 902 when the staple 902 is deployed.

When a user has removed enough bone and shaped the first bone 910 and/or second bone 912 to a desired contour, the user may reposition the inserter 904 and staple 902 for deployment. Next, the user can advance the staple 902 until the bottom surface 914 contacts the newly shaped surface of the first bone 910 and second bone 912, The user is assured that that staple 902 will sit flush and secure against both bones.

In certain embodiments, a rasp blade having reduced overall dimensions, such as a shorter body length, a narrower width, a shorter foot length, and/or a narrower foot width, may be particularly advantageous for minimally invasive surgery (MIS). MIS techniques generally seek to limit the size of incisions required to perform a surgical procedure, thereby reducing soft-tissue disruption, wound healing time, post-operative pain and the risk of infection. A smaller sized rasp blade can be more easily introduced and manipulated through limited surgical access portals, small incisions, or minimally invasive working channels while still providing effective bone contouring, shaping, or surface preparation. In such embodiments, the rasp blade may retain all functional features described herein, including compatibility with powered oscillation handpieces, rasp features configured for controlled bone removal and/or debris evacuation structures, while being scaled to dimensions suitable for MIS applications. These smaller embodiments may be particularly useful in procedures involving confined anatomical spaces.

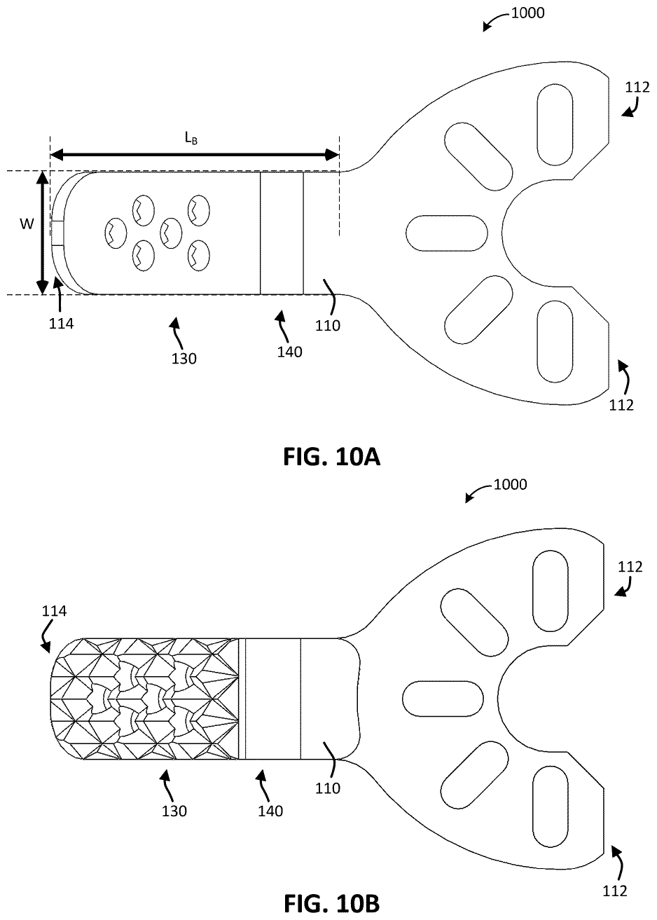

FIGS. 10A and 10B illustrate a superior view and an inferior view, respectively of an embodiment for a rasp blade 1000. The rasp blade 1000 may include many of the same structures, features, functions, operational characteristics, and configurations as the rasp blade 100 described with reference to FIGS. 1-3B. Like parts are identified with the same reference numerals. Accordingly, the rasp blade 1000 may include a body 110, a proximal end 112, distal end 114, a foot 130, angle feature 140, and rasp features 136.

In the illustrated embodiment, the rasp blade 1000 has a smaller width W and a shorter length L_{B} than the rasp blade 100. The oscillation mount 120 may remain generally consistent with the other disclosed embodiments such that the oscillation mount 120 remains compatible with one or more conventional powered oscillation handpieces. One or more of the body 110, the angle feature 140, and/or the foot 130 may have a smaller length L_{B} and/or width W than the corresponding portions of embodiments such as the rasp blade 100. For example, and without limitation, one or more of the body 110, the angle feature 140, and/or the foot 130 may have a width in a range from about 3 mm to about 10 mm. In certain embodiments, the width may be from about 4 mm to about 8 mm, and in one embodiment the width may be about 5 mm.

The length of the portion of the rasp blade 1000 extending from the oscillation mount 120 to the distal end 114 may vary and is not intended to be limiting. The distance between the proximal end 112 and the distal end 114, including the length of the body 110 and any associated foot 130, may be selected based on surgical preference, anatomical location, target bone size, or access constraints. Accordingly, the body 110 may be relatively short, intermediate, or elongated as compared to the embodiments illustrated herein, without departing from the scope of the present disclosure. Variations in each or all of body 110, angle feature 140 and foot 130 length may be implemented while maintaining the same functional features and oscillation mount configurations described herein. The illustrated dimensions and example lengths are provided for explanatory purposes and may vary in other embodiments.

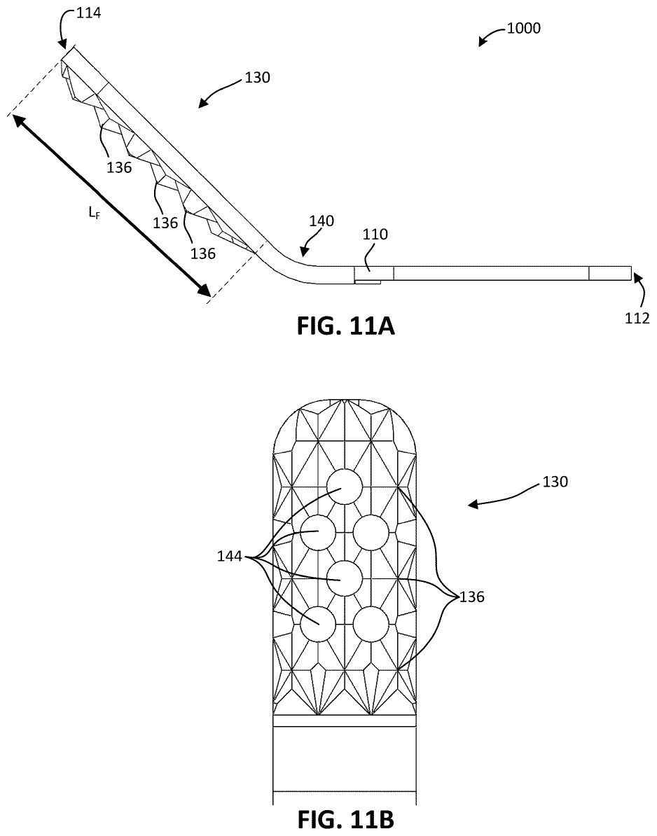

FIG. 11A illustrates a side view of an embodiment for a rasp blade 1000. Accordingly, the rasp blade 1000 may include a body 110, a proximal end 112, distal end 114, a foot 130, angle feature 140, and rasp features 136. As discussed above, the length L_{F} of foot 130 may be relatively short as compared to the embodiments illustrated herein, without departing from the scope of the present disclosure.

FIG. 11B illustrates a plan view of the inferior side 134 of the foot 130 of the rasp blade 1000. In the illustrated embodiment, the openings 144 are circular and may have a substantially uniform diameter and/or spacing. Of course, in other embodiments the openings 144 can have a variety of sizes and/or shapes and can be distributed according to different patterns. In an embodiment, at least one opening 144 of the plurality of openings 144 is disposed between at least two rasp features 136.

A person of ordinary skill in the art will readily appreciate that the length and/or width of the body 110 and the foot 130 may be selected from a variety of dimensions depending on the particular application for which the rasp blade is intended. For example, variations in body length and width and foot length and width may be influenced by factors such as the anatomical location being treated, the size and geometry of the target bone, the degree of surgical access available, surgeon preference. Accordingly, the body and foot may be provided in a variety of shorter, longer, wider, and narrower configurations, and with smaller or larger footprints, while maintaining the functional features and operational principles described herein. Such dimensional variations remain within the scope of the present disclosure. In certain embodiments, the rasp blade may be scaled and/or proportioned as needed to accommodate different surgical techniques, patient anatomies, surgeon preferences, and/or procedural requirements.

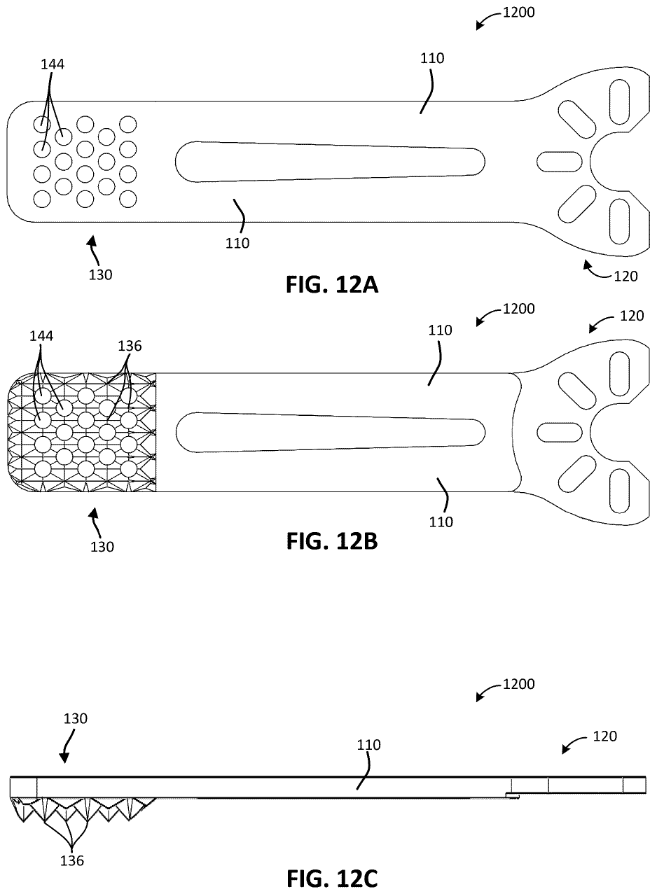

FIGS. 12A-12C illustrate views of an embodiment of a rasp blade 1200 that is substantially flat throughout its entire length. FIG. 12A illustrates a top or superior side view of an embodiment of a rasp blade 1200. FIG. 12B illustrates a bottom or inferior side view and FIG. 12C illustrates a side view. In this embodiment, rasp blade 1200 may include many of the same structures, features, materials, dimensions, and functional characteristics described herein with respect to the other embodiments, except that rasp blade 1200 may not include an angle feature and/or an inclination curve. Like parts are identified with the same reference numerals.

Instead of including an angle feature and/or an inclination curve, body 110 and foot 130 may be substantially coplanar. The substantially flat configuration can enable controlled bone removal while maintaining compatibility with powered oscillation handpieces and providing predictable engagement between the rasp features and a target bone surface. In this embodiment, debris evacuation features, such as openings 144 extending through the foot 130 and/or rasp feature 136 configurations including coarse and/or fine rasp features may be included as described in other embodiments. The substantially flat rasp blade 1200 may be particularly suited for applications where access geometry favors a linear approach to the bone surface or where a planar working profile is desired without angular offset, while still enabling precise surface contouring, smoothing, and preparation of bone surfaces for fixation or implant placement.

Those of skill in the art will appreciate that embodiments of the system disclosed herein can be used on humans and on bones that are relatively small in comparison to other bones of the body (e.g., bones of the foot and hand). Advantageously, the embodiments of the system seek to minimize the number of fasteners or pins placed within the bones of a patient by planning a surgical procedure such that pins or fasteners placed in one stage are and/or can be reused in subsequent stages. Consequently, pins initially deployed can remain in the bone or bone fragment as instruments are deployed and/or subsequent stages of the surgical procedure are performed.

Advantageously, because the present disclosure uses a bone model of the patient's bones the sizes, dimensions, lengths and configurations of the components of the example systems can each be changed, adapted, revised, and/or customized to meet the needs and/or preferences of the patient and/or surgeon. Advantageously, using the apparatus, systems, and/or methods of the present disclosure the surgeon may have a preoperative plan that identifies which specific bone screw (length, width, diameter, thread, pitch, etc.) to use for the fasteners.

Advantageously, the present disclosure provides an apparatus, system, and/or method that can remediate a condition in a patient's foot. Those of skill in the art will appreciate that the methods, processes, apparatuses, systems, devices, and/or instruments of the present disclosure can be used to address a variety of conditions in a variety of procedures and/or parts of the body of the patient.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it in spirit and scope.

1. A rasp blade for shaping a bone surface, the rasp blade comprising:
a body having a proximal end, a distal end, and a longitudinal axis that extends from the proximal end to the distal end;
an oscillation mount at the proximal end, the oscillation mount configured to couple the rasp blade to a powered oscillation handpiece;
a foot comprising:
a superior side; and
a planar inferior side comprising a plurality of rasp features; and
an angle feature that connects the foot and the body at an acute angle relative to the longitudinal axis of the body.

2. The rasp blade of claim 1, wherein the superior side of the foot is planar and the foot comprises a plurality of openings that extend from the superior side to the inferior side, the plurality of openings configured to facilitate evacuation of bone debris.

3. The rasp blade of claim 2, wherein at least one opening of the plurality of openings is disposed between at least two rasp features.

4. The rasp blade of claim 3, wherein the acute angle comprises an inclination angle relative to the longitudinal axis of the body, the inclination angle within the range of about 30 degrees to about 60 degrees.

5. The rasp blade of claim 4, wherein the inclination angle relative to the longitudinal axis of the body is 45 degrees.

6. The rasp blade of claim 1, wherein the superior side of the foot is planar.

7. The rasp blade of claim 1, wherein the angle feature comprises an inclination curve that connects the body and the foot.

8. The rasp blade of claim 1, wherein the oscillation mount has an oscillation mount thickness and the body has a body thickness greater than the oscillation mount thickness.

9. The rasp blade of claim 1, wherein at least the body and the foot are configured to provide a mass moment of inertia at the plurality of rasp features of between about 725 g*mm\hat{ }2 to 750 g*mm\hat{ }2.

10. The rasp blade of claim 1, wherein the body comprises a shaped opening between the foot and the oscillation mount.

11. The rasp blade of claim 10, wherein the shaped opening comprises a proximal end and a distal end and the distal end has a larger diameter than the proximal end.

12. The rasp blade of claim 1, wherein the rasp blade comprises a pivot axis perpendicular to the longitudinal axis and the body and the foot define a moment arm of between about 25 mm and about 40 mm.

13. A surface contouring blade for contouring a bone surface, the surface contouring blade comprising:
a body having a proximal end, a distal end, and longitudinal axis that extends from the proximal end to the distal end;
an oscillation mount at the proximal end, the oscillation mount configured to couple the surface contouring blade to a powered oscillation handpiece;
wherein the distal end of the body comprises:
a superior side; and
a planar inferior side comprising a plurality of teeth; and
an inclination feature that tilts the distal end of the body at an acute angle relative to the longitudinal axis.

14. The surface contouring blade of claim 13, wherein the inclination feature comprises an inclination curve that connects the distal end of the body to the body, the inclination curve configured to bias the distal end to return to the acute angle in response to pressing the plurality of teeth against a bone surface.

15. The surface contouring blade of claim 13, wherein at least one tooth of the plurality of teeth comprises a base connected to a tip by four sides;
wherein a first side extends from the base and is adjacent to a second side;
the second side extends from the base and is adjacent to a third side;
the third side extends from the base and is adjacent to a fourth side;
the fourth side extends from the base and is adjacent to the third side and the first side;
wherein each of the four sides converge at the tip to form an abrading feature.

16. The surface contouring blade of claim 15, wherein:
the first side meets the second side at the base at an obtuse angle;
the second side meets the third side at the base at an acute angle;
the third side meets the fourth side at the base at an obtuse angle; and
the fourth side meets the first side at the base at an acute angle.

17. The surface contouring blade of claim 13, wherein the plurality of teeth comprises a coarse rasp feature and a fine rasp feature.

18. The surface contouring blade of claim 17, wherein the coarse rasp feature comprises a set of teeth having at least one of a tooth height and a tooth separation distance that is greater than a corresponding at least one of a tooth height and a tooth separation distance for a set of teeth of the fine rasp feature.

19. A method for remediating a bone condition present in a patient's foot, the method comprising:
determining a need for surface contouring of one or more bones of the patient in a surgical procedure;
coupling a powered oscillation handpiece to a rasp blade, the rasp blade comprising:
a body having a proximal end, a distal end, and longitudinal axis that extends from the proximal end to the distal end;
an oscillation mount at the proximal end, the oscillation mount configured to couple the rasp blade to a powered oscillation handpiece;
a foot comprising:
a superior side; and
a planar inferior side comprising a plurality of rasp features; and
an angle feature that connects the foot and the body at an acute angle relative to the longitudinal axis of the body;
activating the powered oscillation handpiece;
positioning the plurality of rasp features against a surface of one or more bones of the patient; and
removing a controlled amount of bone from the one or more bones.

20. The method of claim 19, further comprising, exchanging a first rasp blade coupled to the powered oscillation handpiece with a second rasp blade, the first rasp blade comprising a coarse rasp feature and the second rasp blade comprising a fine rasp feature.
